SEQUENCE LISTING (6 sequences)

SEQ ID NO: 1 - protein, 1255 aa, SARS Coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 2 - protein, 1203 aa, Artificial Sequence
  Ssol amino acid sequence
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKSGDYKDDDDK

SEQ ID NO: 3 - DNA, 3783 bp, Artificial Sequence
  DNA sequence encoding S protein, inserted within a BamH1-Xho1 cassette
ggatccaccatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtctctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaatccatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatgggcttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggctaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataactcgag

SEQ ID NO: 4 - DNA, 3627 bp, Artificial Sequence
  DNA sequence encoding Ssol polypeptide, inserted within a BamH1-Xho1 cassette
ggatccaccatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtctctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaatccatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatgggcttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggctaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatccggagattataaagatgacgacgataaataactcgag

SEQ ID NO: 5 - protein, 8 aa, Artificial Sequence
  Octapeptide Flag
DYKDDDDK

SEQ ID NO: 6 - protein, 10 aa, Artificial Sequence
  Serine-Glycine linker and octapeptide Flag
SGDYKDDDDK

(19) United States
(12) Patent Application Publication
(10) Pub. No.: US 2012/0045469 A1
(43) Pub. Date: Feb. 23, 2012

(54) VACCINE

(75) Inventors: Benoit Baras, Rixensart (BE); Benoit Callendret, Paris (FR); Nicolas Escriou, Paris (FR); Valerie Lorin, Paris (FR); Philippe Marianneau, Lyon (FR); Sylvie Van Der Werf, Paris (FR); Martine Anne Cecile Wettendorff, Rixensart (BE)

(73) Assignee: GlaxoSmithKline Biologicals S.A., Rixensart (BE)

(21) Appl. No.: 13/132,364

(22) PCT Filed: Dec. 1, 2009
(86) PCT No.: PCT/EP2009/066089

§ 371 Date: Nov. 14, 2011

(30) Foreign Application Priority Data
Dec. 2, 2008 (GB) 08220014

(51) Int. Cl. A61K 39/12; A61P 37/04; A61P 31/12

(52) U.S. Cl. 424/186.1

(57) ABSTRACT

The present invention provides an immunogenic composition comprising: an immunogenic SARS coronavirus S (spike) polypeptide, or a fragment or variant thereof; and an adjuvant comprising an oil-in-water emulsion.

### FIELD OF THE INVENTION

The present invention relates to vaccines against severe acute respiratory syndrome coronavirus (SARS-CoV) infection, and their use in the prevention of SARS. The invention also relates to methods of producing such vaccines.

### BACKGROUND TO THE INVENTION

Coronavirus has a positive-sense, non-segmented, single-stranded RNA genome, which encodes at least 18 viral proteins including the structural proteins E, M, N and S. The S (spike) protein, a major antigen of coronavirus, is a membrane glycoprotein (200-220 kDa) which exists in the form of spikes emerging from the surface of the viral envelope. It is responsible for the attachment of the virus to the receptors of the host cell and for inducing the fusion of the viral envelope with the cell membrane. The S protein has two domains: S1, which is believed to be involved in receptor binding; and S2, believed to mediate membrane fusion between the virus and target cell (Holmes and Lai, 1996). The S protein can form non-covalently linked homotrimers (oligomers), which may mediate receptor binding and virus infectivity.

In March 2003, a new coronavirus (SARS-CoV or SARS virus) was isolated, in association with cases of severe acute respiratory syndrome (SARS). Genomic sequences of this new coronavirus have been obtained, including those of the Urbani isolate (Genbank accession No. AY274119.3 and A. MARRA et al., Science, May 1, 2003, 300, 1399-1404) and the Toronto isolate (Tor2, Genbank accession No. AY278741 and A. ROTA et al., Science, 2003, 300, 1394-1399).

Another strain of SARS-associated coronavirus has also been identified, which is distinguishable from the Tor2 and Urbani isolates. This coronavirus strain is derived from the sample collected from the bronchoaleveolar washings from a patient suffering from SARS, recorded under the No. 031589 and collected at the Hanoi (Vietnam) French hospital (WO 2005/056781 and WO 2005/056584).

### SUMMARY OF THE INVENTION

The present invention provides an immunogenic composition comprising an immunogenic SARS coronavirus S (spike) polypeptide, or a fragment or variant thereof, and an oil-in-water emulsion adjuvant. The invention also provides a method of producing an immunogenic composition of the invention, the method comprising combining an immunogenic S polypeptide, or a fragment or variant thereof, with an oil-in-water emulsion adjuvant.

The invention further provides:
an immunogenic composition of the invention for use as a medicament;
an immunogenic composition of the invention for the prevention or treatment of severe acute respiratory syndrome or other SARS-CoV-related disease;
use of an immunogenic composition of the invention for the manufacture of a medicament for the prevention or treatment of severe acute respiratory syndrome or other SARS-CoV-related disease;
a method of preventing or treating severe acute respiratory syndrome or other SARS-CoV-related disease, which method comprises administering an effective amount of an immunogenic composition of the invention to an individual in need thereof; and
an immunogenic composition of the invention for use as a vaccine.

### BRIEF DESCRIPTION OF THE DRAWINGS

FIG. 1 shows the effect of adjuvants on the humoral response induced by the Ssol polypeptide. Young adult BALB/c mice (8 per group) were immunised, at three week intervals, by two intramuscular injections of 2 μg of Ssol protein without adjuvant (no adj.) or associated with 50 μg of Alum or with 50 μL of the oil-in-water emulsion adjuvant (GSK2 adj). Two control groups were immunised with each of the adjuvants alone. The sera were collected three weeks after each injection (IS1 and IS2, respectively), and the specific antibody response to the SARS-CoV native antigens measured by anti-SARS ELISA as described in Callendret et al. (Virology, 2007, 363 : 288-302). The titers from each mouse are represented by black dots, and the averages by horizontal bars. The detection limit of the experiment is represented by a dotted line.

FIG. 2 shows the effect of adjuvants on the neutralising humoral response induced by the Ssol polypeptide. Young adult BALB/c mice (8 per group) were immunised as described above. The neutralising antibody titers of sera collected three weeks after the last injection were measured as described in Callendret et al. (Virology, 2007, 363 : 288-302). The titers from each mouse are represented by dots, and the averages by horizontal bars. The detection limit of the experiment is represented by a dotted line.

FIG. 3 shows modulation of the immune response type induced by the Ssol protein in the BALB/c mouse by using adjuvants. The specific IgG1 and IgG2a isotype titers to the SARS-CoV native antigens were measured on the mice sera collected 3 weeks after the last immunisation. The titers measured for each mouse are shown as dots. For the control groups, the titers were measured on the mix of sera from each group, and shown by a diamond shape. The detection limit of the experiment is shown by a dotted line.

FIG. 4 shows the effect of adjuvants on the humoral response induced by the Ssol polypeptide in Syrian Golden hamsters. The sera were collected three weeks after each injection (IS1 and IS2, respectively) and three months after the second injection (IS2bis), and the specific antibody response to the SARS-CoV native antigens measured by anti-SARS ELISA as in FIG. 1. The titers from each hamster are represented by black dots and the averages by horizontal bars.

FIG. 5 shows the effect of adjuvants on the neutralising humoral response induced by the Ssol polypeptide in Syrian Golden hamsters. The neutralising antibody titers of sera collected three months after the last injection were measured as described in FIG. 2. The titers from each hamster are represented by dots and the averages by horizontal bars.

FIGS. 6 and 7 show the effect of adjuvants on the protective immune response induced by the Ssol polypeptide in Syrian Golden hamsters. Three months after the second injection, hamsters were challenged intranasally with 10⁵ PFU of SARS-CoV. Four days after inoculation, hamsters were euthanized. Lungs and upper respiratory tract (URT, i.e. pharynx plus trachea) homogenates were prepared and titrated for infectious SARS-CoV by plaque assay on Vero cells, as described in Callendret et al. (Virology, 2007, 363 : 288-302). Values for each individual hamster are represented with black circles for lung (FIG. 6) and URT (FIG. 7), and means with horizontal bars. The detection limits of the assays are indicated by a dotted line.

FIG. 8 shows the results of histopathological analysis of the lungs of challenged hamsters previously immunized with 0.2 μg of Ssol protein. The scores of pulmonary inflammation and lesions (HE) and the scores of viral antigen loads (IHC) are shown on a 1-10 scale.

FIG. 9 shows SARS-CoV specific IgG antibody titers determined by indirect ELISA from serum obtained on day 14 post-immunization from BALB/c mice immunized with different doses of Ssol, alone or adjuvanted with Alum or the oil-in-water emulsion adjuvant (GSK2 adjuvant).

FIG. 10 shows SARS-CoV isotype antibody titers determined by indirect ELISA from serum obtained on day 14 post-immunization from BALB/c mice immunized with 2 μg of Ssol, alone or adjuvanted with Alum or the oil-in-water emulsion adjuvant (GSK2 adjuvant).

FIG. 11 shows SARS-CoV neutralizing antibody titers determined from serum obtained on day 14 post-immunization from BALB/c mice immunized with 0.2 μg of Ssol, alone or adjuvanted with Alum or the oil-in-water emulsion adjuvant (GSK2 adjuvant).

FIG. 12 shows CD4+ T cell response in PBMC obtained on day 7 post-immunization from BALB/c mice immunized with different doses of Ssol, alone or adjuvanted with Alum or oil-in-water emulsion (GSK2 adj).

FIG. 13 shows CD4+ T cell response in spleen obtained on day 14 post-immunization from BALB/c mice immunized with different doses of Ssol, alone or adjuvanted with Alum or oil-in-water emulsion (GSK2 adj).

FIG. 14 shows cytokine secretion (IL-5, IL-13 and IFN-γ) from spleen cells obtained on day 14 post-immunization from BALB/c mice immunized with different doses of Ssol, alone or adjuvanted with Alum or oil-in-water emulsion (GSK2 adj).

FIG. 15 shows SARS-CoV specific IgG antibody titers determined by indirect ELISA from serum obtained on day 14 post-immunization from C57BL/6 mice immunized with different doses of Ssol, alone or adjuvanted with Alum or the oil-in-water emulsion adjuvant (GSK2 adjuvant).

FIG. 16 shows SARS-CoV isotype antibody titers determined by indirect ELISA from serum obtained on day 14 post-immunization from C57BL/6 mice immunized with 2 μg of Ssol, alone or adjuvanted with Alum or the oil-in-water emulsion adjuvant (GSK2 adjuvant).

FIG. 17 shows SARS-CoV neutralizing antibody titers determined from serum obtained on day 14 post-immunization from C57BL/6 mice immunized with 0.2 μg of Ssol, alone or adjuvanted with Alum or the oil-in-water emulsion adjuvant (GSK2 adjuvant).

FIG. 18 shows CD4+ T cell response in PBMC obtained on day 7 post-immunization from C57B1/6 mice immunized with different doses of Ssol, alone or adjuvanted with Alum or oil-in-water emulsion (GSK2 adj).

FIG. 19 shows CD4+ T cell response in spleen cells obtained on day 14 post-immunization from C57B1/6 mice immunized with different doses of Ssol adjuvanted with oil-in-water emulsion (GSK2 adj).

FIG. 20 shows cytokine secretion (IL-5, IL-13 and IFN-γ) from spleen cells obtained on day 14 post-immunization from C57B1/6 mice immunized with different doses of Ssol adjuvanted with oil-in-water emulsion (GSK2 adj).

FIG. 21 shows the effect of adjuvants on the neutralising humoral response induced by 2 μg of the Ssol polypeptide in Syrian Golden hamsters. The neutralising antibody titers of sera collected eight months after the last injection were measured as described in FIG. 2. The titers from each hamster are represented by dots and the averages by horizontal bars.

FIGS. 22 and 23 show the effect of adjuvants on the protective immune response induced by 2 μg of the Ssol polypeptide in Syrian Golden hamsters. Eight months after the second injection, hamsters were challenged intranasally with 10⁵ PFU of SARS-CoV. Four days after inoculation, hamsters were euthanized. Lungs and upper respiratory tract (URT, i.e. pharynx plus trachea) homogenates were prepared and titrated for infectious SARS-CoV by plaque assay on Vero cells, as described in Callendret et al. (Virology, 2007, 363 : 288-302). Values for each individual hamster are represented with black circles for lung (FIG. 22) and URT (FIG. 23), and means with horizontal bars. The detection limits of the assays are indicated by a dotted line.

FIG. 24 shows the results of histopathological analysis of the lungs of challenged hamsters previously immunized with 2 μg of Ssol protein. The scores of pulmonary inflammation and lesions (HE) and the scores of viral antigen loads (IHC) are shown on a 1-10 scale.

FIG. 25 shows the effect of adjuvants on the humoral response induced by a single injection of 0.2 μg of the Ssol polypeptide in Syrian Golden hamsters. The sera were collected two weeks after the injection, and the specific antibody response to the SARS-CoV native antigens measured by anti-SARS ELISA as in FIG. 1. The titers from each hamster are represented by black dots and the averages by horizontal bars.

FIG. 26 shows the effect of adjuvants on the neutralising humoral response induced by a single injection of 0.2 μg of the Ssol polypeptide in Syrian Golden hamsters. The neutralising antibody titers of sera collected two weeks after the injection were measured as described in FIG. 2. The titers from each hamster are represented by dots and the averages by horizontal bars.

FIGS. 27 and 28 show the effect of adjuvants on the protective immune response induced by a single injection of 0.2 μg of the Ssol polypeptide in Syrian Golden hamsters. Three weeks after the injection, hamsters were challenged intranasally with 10⁵ PFU of SARS-CoV. Four days after inoculation, hamsters were euthanized. Lungs and upper respiratory tract (URT, i.e. pharynx plus trachea) homogenates were prepared and titrated for infectious SARS-CoV by plaque assay on Vero cells, as described in Callendret et al. (Virology, 2007, 363 : 288-302). Values for each individual hamster are represented with black circles for lung (FIG. 27) and URT (FIG. 28), and means with horizontal bars. The detection limits of the assays are indicated by a dotted line.

FIG. 29 shows the results of histopathological analysis of the lungs of challenged hamsters previously immunized with a single injection of 0.2 μg of Ssol protein. The scores of pulmonary inflammation and lesions (HE) and the scores of viral antigen loads (IHC) are shown on a 0-5 scale.

### DETAILED DESCRIPTION

The present invention provides an immunogenic composition which is useful in the prevention or treatment of severe acute respiratory syndrome (SARS) or other SARS-CoV-related disease. The term “immunogenic composition”, as used in the present invention, refers to a composition that comprises an immunogenic component capable of provoking an immune response in an individual, such as a human, optionally when suitably formulated with an adjuvant. Accordingly, in one embodiment the invention provides an immunogenic composition comprising an immunogenic SARS coronavirus S (spike) polypeptide, or a fragment or variant thereof, and an oil-in-water emulsion adjuvant. In another embodiment of the invention, the immunogenic composition of the invention is a vaccine, i.e. the immunogenic composition is capable of provoking a protective immune response against a SARS-CoV infection.

The immunogenic composition of the present invention comprises immunogenic SARS coronavirus S (spike) polypeptides, including fragments and variants thereof. The immunogenic S polypeptides may comprise any portion of an S protein that has an epitope capable of eliciting a protective immune response, for example an epitope capable of eliciting production of a neutralizing antibody and/or stimulating a cell-mediated immune response, against a SARS-CoV infection.

An exemplary SARS-CoV S protein has 1,255 amino acids (see for example SEQ ID NO:1), with a 13 amino acid signal sequence, the 51 domain at amino acids 12-672, and the S2 domain at amino acids 673-1192. The protein consists of a signal peptide (amino acids 1-13), an extracellular domain (amino acids 14-1195), a transmembrane domain (amino acids 1196-1218) and an intracellular domain (amino acids 1219-1255). The S protein sequence may be derived from any SARS-CoV strain, including those known to have caused SARS in human populations, for example the Tor2, Urbani or No. 031589 strains, or from any other strain of SARS-CoV, for example a strain that is circulating in an animal population, such as civets or bats, that has not yet entered the human population.

In one embodiment, the immunogenic S polypeptide is a portion or fragment of the full-length S protein. As described herein, an immunogenic S polypeptide includes a fragment of S protein or a S protein variant (which may be a variant of a full-length S protein or S fragment as described herein) that has at least one epitope contained within the full-length S protein or wildtype S protein, respectively, that elicits a protective immune response against SARS coronavirus.

In one embodiment, the immunogenic S polypeptide may consist of or comprise the entire extracellular domain (ectodomain) of the S protein, for example amino acids 1 to 1193. Hence, the immunogenic S polypeptide may consist of the S glycoprotein with its intracytoplasmic and transmembrane domains deleted.

Optionally, the signal peptide (amino acids 1 to 13) may be deleted. In one embodiment, the immunogenic S polypeptide consists of the extracellular domain of the S protein extended to its C-terminus by a Serine-Glycine linker (SG) and octapeptide Flag (DYKDDDDK). In particular, the immunogenic S polypeptide may consist of or comprise amino acids 14 to 1193 of the SARS-CoV S protein fused at the C-terminal to the sequence SGDYKDDDDK. In a further embodiment, the S polypeptide may consist of or comprise the sequence of SEQ ID NO: 2.

An S protein fragment that comprises an epitope that stimulates, induces, or elicits an immune response may comprise a sequence of consecutive amino acids ranging from any number of amino acids between 8 amino acids and 150 amino acids (e.g., 8, 10, 12, 15, 18, 20, 25, 30, 35, 40, 50, etc. amino acids) of SEQ ID NO: 1.

In other embodiments, a coronavirus S polypeptide variant has at least 50% to 100% amino acid identity (that is, at least 50%, 55%, 60%, 65%, 70%, 75%, 80%, 85%, 90%, 95%, or 99% identity) to the amino acid sequence of the full length S protein as set forth in SEQ ID NO: 1. Such S polypeptide variants and fragments may retain at least one S protein-specific biological activity or function, such as: (1) the capability to elicit a protective immune response, for example, a neutralizing response and/or a cell-mediated immune response against SARS-CoV; (2) the capability to mediate viral infection via receptor binding; and (3) the capability to mediate membrane fusion between a virion and the host cell.

An S polypeptide may contain conservative amino acid substitutions. Examples of conservative substitutions include substituting one aliphatic amino acid for another, such as Ile, Val, Leu, or Ala, or substituting one polar residue for another, such as between Lys and Arg, Glu and Asp, or Gln and Asn. A similar amino acid or a conservative amino acid substitution is also one in which an amino acid residue is replaced with an amino acid residue having a similar side chain, which include amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, histidine); nonpolar side chains (e.g., alanine, valine, leucine, isoleucine, proline, phenylalanine, methionine, tryptophan); beta-branched side chains (e.g., threonine, valine, isoleucine), and aromatic side chains (e.g., tyrosine, phenylalanine, tryptophan). Proline, which is considered more difficult to classify, shares properties with amino acids that have aliphatic side chains (e.g., Leu, Val, Ile, and Ala). In certain circumstances, substitution of glutamine for glutamic acid or asparagine for aspartic acid may be considered a similar substitution in that glutamine and asparagine are amide derivatives of glutamic acid and aspartic acid, respectively.

Conservative and similar substitutions of amino acids in the coronavirus immunogen sequences disclosed herein may be readily prepared according to methods described herein and practiced in the art and which provide variants retaining similar physical properties and functional or biological activities, such as, for example, the capability to induce or elicit an immune response, which may include a humoral response (that is, eliciting antibodies that bind to and have the same biological activity as an antibody that specifically binds to the wildtype (or nonvariant) immunogen and/or that binds to antibodies that specifically bind to the wildtype or nonvariant immunogen). An S protein immunogen variant thereof may, for example, retain the capability to bind to cellular receptors and to mediate infectivity.

As used herein, “percent identity” or “% identity” is the percentage value returned by comparing the whole of the subject polypeptide, peptide, or variant thereof sequence to a test sequence using a computer implemented algorithm, typically with default parameters. The variant immunogens described herein could be made to include one or more of a variety of mutations, such as point mutations, frameshift mutations, missense mutations, additions, deletions, and the like, or the variants can be a result of modifications, such as by certain chemical substituents, including glycosylation and alkylation.

As described herein, S protein immunogens, fragments, and variants thereof described herein contain an epitope that elicits or induces an immune response, for instance a protective immune response, which may be a humoral response and/or a cell-mediated immune response. A protective immune response may be manifested by at least one of the following: preventing infection of a host by a coronavirus; modifying or limiting the infection; aiding, improving, enhancing, or stimulating recovery of the host from infection; and generating immunological memory that will prevent or limit a subsequent infection by a SARS coronavirus. In case of SARS CoV infection, the protective immune response can be assessed for instance by the viral load in lungs and upper respiratory tract, the score of pulmonary inflammation and lesions, the scores of viral antigen loads in lungs, the presence of seric neutralizing antibodies, the CD4+ T cell responses in PBMC, spleen and the cytokine secretion from spleen. A humoral response may include production of antibodies that neutralize infectivity, lyse the virus and/or infected cell, facilitate removal of the virus by host cells (for example, facilitate phagocytosis), and/or bind to and facilitate removal of viral antigenic material. A humoral response may also include a mucosal response, which comprises eliciting or inducing a specific mucosal IgA response.

Induction of an immune response in a subject or host (human or non-human animal) by a SARS-CoV S polypeptide, fragment, or variant described herein, may be determined and characterized by methods described herein and routinely practiced in the art. These methods include in vivo assays, such as animal immunization studies, for example, using a rabbit, mouse, ferret, civet cat, African green monkey, or rhesus macaque model, and any one of a number of in vitro assays, such as immunochemistry methods for detection and analysis of antibodies, including Western immunoblot analysis, ELISA, immunoprecipitation, radioimmunoassay, and the like, and combinations thereof.

Other methods and techniques that may be used to analyze and characterize an immune response include neutralization assays (such as a plaque reduction assay or an assay that measures cytopathic effect (CPE) or any other neutralization assay practiced by persons skilled in the art). These and other assays and methods known in the art can be used to identify and characterize S protein immunogens and variants thereof that have at least one epitope that elicits a protective humoral or cell-mediated immune response against SARS coronavirus. The statistical significance of the results obtained in the various assays may be calculated and understood according to methods routinely practiced by persons skilled in the relevant art.

The coronavirus S protein immunogens (full-length proteins, variants, or fragments thereof), as well as corresponding nucleic acids encoding such immunogens, are provided in an isolated form, and in certain embodiments, are purified to homogeneity. As used herein, the term “isolated” means that the nucleic acid or polypeptide is removed from its original or natural environment.

A SARS coronavirus S protein immunogen and fragments and variants thereof may be produced synthetically or recombinantly. A coronavirus protein fragment that contains an epitope that induces an immune response against coronavirus may be synthesized by standard chemical methods, including synthesis by automated procedure. Alternatively, the S protein immunogens may be produced recombinantly. For example, the S protein immunogen may be expressed from a polynucleotide that is operably linked to an expression control sequence, such as a promoter, in a nucleic acid expression construct. For example, the S protein immunogen may be encoded by the DNA sequence of SEQ ID NO: 3 or 4. The SARS coronavirus S polypeptides and fragments or variants thereof may be expressed in mammalian cells, yeast, bacteria, insect or other cells under the control of appropriate expression control sequences. Cell-free translation systems may also be employed to produce such coronavirus proteins using nucleic acids, including RNAs, and expression constructs. Appropriate cloning and expression vectors for use with prokaryotic and eukaryotic hosts are routinely used by persons skilled in the art and are described, for example, by Sambrook et al., Molecular Cloning: A Laboratory Manual, Second Edition, Cold Spring Harbor, N.Y., (1989) and Third Edition (2001), and may include plasmids, cosmids, shuttle vectors, viral vectors, and vectors comprising a chromosomal origin of replication as disclosed therein.

As will be appreciated by those of ordinary skill in the art, a nucleotide sequence encoding a coronavirus S polypeptide or variant thereof may differ from the sequences presented herein due to, for example, the degeneracy of the genetic code. A nucleotide sequence that encodes a coronavirus polypeptide variant includes a sequence that encodes a homologue or strain variant or other variant. Variants may result from natural polymorphisms or may be synthesized by recombinant methodology, for example to introduce an amino acid mutation, or chemical synthesis, and may differ from wild-type polypeptides by one or more amino acid substitutions, insertions, deletions, and the like.

### Adjuvant TH1 and TH2 Immune Responses

An immune response may be broadly divided into two extreme categories, being a humoral or cell mediated immune response (traditionally characterised by antibody and cellular effector mechanisms of protection respectively). These categories of response have been termed TH1-type responses (cell-mediated response), and TH2-type immune responses (humoral response).

Extreme TH1-type immune responses may be characterised by the generation of antigen specific, haplotype restricted cytotoxic T lymphocytes, and natural killer cell responses. In mice TH1-type responses are often characterised by the generation of antibodies of the IgG2a and/or IgG2b subtype, whilst in the human these correspond to IgG1 type antibodies. TH2-type immune responses are characterised by the generation of a range of immunoglobulin isotypes including in mice IgG1.

It can be considered that the driving forces behind the development of these two types of immune responses are cytokines High levels of TH1-type cytokines tend to favour the induction of cell mediated immune responses to the given antigen, whilst high levels of TH2-type cytokines tend to favour the induction of humoral immune responses to the antigen.

The distinction of TH1 and TH2-type immune responses is not absolute, and can take the form of a continuum between these two extremes. In reality an individual will support an immune response which is described as being predominantly TH1 or predominantly TH2. However, it is often convenient to consider the families of cytokines in terms of that described in murine CD4 +ve T cell clones by Mosmann and Coffman (Mosmann, T. R. and Coffman, R. L. (1989) TH1 and TH2 cells: different patterns of lymphokine secretion lead to different functional properties. Annual Review of Immunology, 7, p 145-173). Traditionally, TH1-type responses are associated with the production of the INF-γ cytokines by T-lymphocytes. Other cytokines often directly associated with the induction of TH1-type immune responses are not produced by T-cells, such as IL-12. In contrast, TH2-type responses are associated with the secretion of IL-4, IL-5, IL-6, IL-10 and tumour necrosis factor-β(TNF-β).

It is known that certain vaccine adjuvants are particularly suited to the stimulation of either TH1 or TH2-type cytokine responses. Traditionally indicators of the TH1:TH2 balance of the immune response after a vaccination or infection includes direct measurement of the production of TH1 or TH2 cytokines by T lymphocytes in vitro after restimulation with antigen, and/or the measurement (at least in mice) of the IgG1:IgG2a or IgG1:IgG2b ratio of antigen specific antibody responses.

Thus, a TH1-type adjuvant is one which stimulates isolated T-cell populations to produce high levels of TH1-type cytokines when re-stimulated with antigen in vitro, and induces antigen specific immunoglobulin responses associated with TH1-type isotype.

### Oil-in-Water Emulsion Adjuvant

The immunogenic composition of the invention contains an oil-in-water emulsion adjuvant. Oil-in-water emulsions per se are well known in the art, and have been suggested to be useful as adjuvant compositions (EP 399843; WO 95/17210).

In order for any oil-in-water composition to be suitable for human administration, the oil phase of the emulsion system has to comprise a metabolisable oil. The meaning of the term “metabolisable” is well known in the art, and can be defined as ‘being capable of being transformed by metabolism’ (Dorland's Illustrated Medical Dictionary, W.B. Sanders Company, 25th edition (1974)). The oil may be any vegetable oil, fish oil, animal oil or synthetic oil, which is not toxic to the recipient and is capable of being transformed by metabolism. Nuts, seeds, and grains are common sources of vegetable oils. Synthetic oils are also part of this invention and can include commercially available oils such as NEOBEE® and others.

A suitable metabolisable oil is squalene (2,6,10,15,19,23-Hexamethyl-2,6,10,14,18,22-tetracosahexaene), an unsaturated oil which is found in large quantities in shark-liver oil, and in lower quantities in olive oil, wheat germ oil, rice bran oil and yeast. Squalene is a metabolisable oil by virtue of the fact that it is an intermediate in the biosynthesis of cholesterol. Suitably the metabolisable oil is present in an amount of 0.5% to 10% (v/v) of the total volume of the immunogenic composition.

The oil-in-water emulsion adjuvant further comprises an emulsifying agent. The emulsifying agent may suitably be polyoxyethylene sorbitan monooleate (Tween 80™). The emulsifying agent is suitably present in the adjuvant composition in an amount of 0.125 to 4% (v/v) of the total volume of the immunogenic composition.

The oil-in-water emulsion of the present invention optionally further comprises a tocol. Tocols are well known in the art and are described in EP0382271. A suitable tocol is alpha-tocopherol or a derivative thereof such as alpha-tocopherol succinate (also known as vitamin E succinate). The tocol is suitably present in the adjuvant composition in an amount of 0.25% to 10% (v/v) of the total volume of the immunogenic composition.

In an oil-in-water emulsion, the oil and emulsifier should be in an aqueous carrier. The aqueous carrier may be, for example, phosphate buffered saline (PBS).

In one embodiment of the invention, the oil-in-water emulsion adjuvant comprises squalene, polyoxyethylene sorbitan monooleate (Tween 80™) and alpha-tocopherol. Typically the oil-in-water emulsion adjuvant will comprise from 2 to 10% squalene, from 0.3 to 3% polyoxyethylene sorbitan monooleate and from 2 to 10% alpha-tocopherol of the total volume of the immunogenic composition, and may be produced according to the procedure described in WO 95/17210. The ratio of squalene:alpha-tocopherol may be equal to or less than 1 as this provides a more stable emulsion. The oil-in-water emulsion may also contain polyoxyethylene sorbitan trioleate (Span 85) and/or Lecithin, for example at a level of 1% of the total volume of the immunogenic composition.

Methods of producing oil-in-water emulsions are well known to the person skilled in the art. Commonly, the method comprises mixing the oil phase (optionally comprising a tocol) with a surfactant such as a PBS/TWEEN80™ solution, followed by homogenisation using a homogenizer. A method comprising passing the mixture twice through a syringe needle would be suitable for homogenising small volumes of liquid. Equally, the emulsification process in microfluidiser (M110S Microfluidics machine, maximum of 50 passes, for a period of 2 minutes at maximum pressure input of 6 bar (output pressure of about 850 bar)) could be adapted by a person skilled in the art to produce smaller or larger volumes of emulsion. The adaptation could be achieved by routine experimentation comprising the measurement of the resultant emulsion until a preparation was achieved with oil droplets of the required diameter.

The oil-in-water emulsion systems of the present invention may have a small oil droplet size in the sub-micron range. Suitably the droplet sizes will be in the range of 120 to 750 nm, for example sizes from 120 to 600 nm in diameter. The oil-in water emulsion may contain oil droplets of which at least 70% by intensity are less than 500 nm in diameter, at least 80% by intensity are less than 300 nm in diameter, or at least 90% by intensity are in the range of 120 to 200 nm in diameter.

The oil droplet size (i.e. diameter) according to the present invention is given by intensity. There are several ways of determining the diameter of the oil droplet size by intensity. Intensity is measured by use of a sizing instrument, suitably by dynamic light scattering such as the Malvern Zetasizer 4000 or the Malvern Zetasizer 3000HS. A first possibility is to determine the z average diameter ZAD by dynamic light scattering (PCS-Photon correlation spectroscopy); this method additionally gives the polydispersity index (PDI), and both the ZAD and PDI are calculated with the cumulants algorithm. These values do not require the knowledge of the particle refractive index. A second means is to calculate the diameter of the oil droplet by determining the whole particle size distribution by another algorithm, either the Contin, or NNLS, or the automatic “Malvern” one (the default algorithm provided for by the sizing instrument). Most of the time, as the particle refractive index of a complex composition is unknown, only the intensity distribution is taken into consideration, and if necessary the intensity mean originating from this distribution.

### Vaccine Formulation and Administration

The amount of the protein of the present invention present in each vaccine dose is selected as an amount which induces an immunoprotective response without significant, adverse side effects in typical vaccines. Such amount will vary depending upon which specific immunogen is employed and the type and amount of adjuvant used. An optimal amount for a particular vaccine may be ascertained by standard studies involving observation of antibody titres and other responses in subjects. Generally, it is expected that each dose will comprise 1-1000 μg of protein, for example 1-200n, or 10-100 μg. A typical dose will contain 10-50 μg, for example 15-25 μg, suitably about 20 μg of protein. Alternatively, a “dose-sparing” approach may be used, for example in a pandemic situation. This is based on the finding that it is possible to provide the same protective effect using lower doses of antigen, due to the presence of an effective adjuvant. Accordingly, each human dose may contain a significantly lower quantity of protein, for example from 0.1 to 10 μg, or 0.5 to 5 μg, or 1 to 3 μg, suitably 2 μg protein per dose. By the term “human dose” is meant a dose which is in a volume suitable for human use. Generally this is between 0.3 and 1.5 ml. In one embodiment, a human dose is 0.5 ml.

Following an initial vaccination, subjects typically receive a boost after a 2 to 4 week interval, for example a 3 week interval, optionally followed by repeated boosts for as long as a risk of infection exists. In a specific embodiment of the invention, a single-dose vaccination schedule is provided, whereby one dose of S protein in combination with adjuvant is sufficient to provide protection against the SARS CoV, without the need for any boost after the initial vaccination.

The immunogenic compositions of the invention may be provided by any of a variety of routes such as oral, topical, subcutaneous, mucosal (typically intravaginal), intraveneous, intramuscular, intranasal, sublingual, intradermal and via suppository.

Immunisation can be prophylactic or therapeutic. The invention described herein is primarily but not exclusively concerned with prophylactic vaccination against SARS.

Appropriate pharmaceutically acceptable carriers or excipients for use in the invention are well known in the art and include for example water or buffers. Vaccine preparation is generally described in Pharmaceutical Biotechnology, Vol. 61 Vaccine Design—the subunit and adjuvant approach, edited by Powell and Newman, Plenum Press New York, 1995. New Trends and Developments in Vaccines, edited by Voller et al., University Park Press, Baltimore, Md., U.S.A. 1978.

The immunogenic compositions of the invention comprise certain components as laid out above. In a further aspect of the invention the immunogenic composition consists essentially of, or consists of, said components.

The present invention is now described with respect to the following examples which serve to illustrate the invention.

### Example 1

### Expression of a Soluble Form of the SARS CoV-S Protein

In order to purify the ectodomain of the S protein in mammalian cells, a gene was constructed enabling the expression of a spike glycoprotein with its intracytoplasmic and transmembrane domains deleted. This polypeptide, called Ssol, comprises the entire extracellular domain of the S protein (amino acids 1-1193) extended to its C-terminus by a Serine-Glycine linker and octapeptide Flag. Since the membrane anchoring domain is deleted, the Ssol polypeptide is secreted into the culture media.

### Constitutive Expression of the Ssol Polypeptide

TRIP lentiviral vectors were used to establish cell lines expressing the Ssol protein in a stable and constitutive way. These vectors are produced by the co-transfection of a pTRIP plasmid vector, a p8.7 packaging plasmid and a pHCMV-VSV-G plasmid (Yee et al., 1994; Zennou et al., 2000; Zufferey et al., 1997).

To construct the TRIP vectors for expression of the Ssol protein, an expression cassette composed of: the CMVi/e promoter, the chimeric intron from pCI plasmid, the Ssol ORF and of one of the two viral export elements CTE or WPRE was transferred into a plasmid pTRIP-EF1-EGFP instead of the EF1 promoter and of the GFP ORF. The plasmids thus produced, called pTRIP-Ssol-CTE and pTRIP-Ssol-WPRE were used to produce TRIP-Ssol-CTE and TRIP-Ssol-WPRE lentiviral vector stocks respectively. These vectors were used to transduce the FRhK-4 cells according to a series of 5 consecutive transduction cycles spaced over 24 hours. The transduced cells were cloned by limiting dilution, and the cell clones obtained selected depending on their marked secretion of the polypeptide Ssol polypeptide. To do this, a fixed number of cells from various clones were seeded in 35 mm culture dishes, and the presence of the Ssol protein in the supernatant analysed by western blot 72 hours later.

A protein of the expected size (˜180 kDa) was detected in the supernatants from all the clones, confirming the efficiency of the transduction protocol. However, the expression levels varied from one clone to the other, independently of the TRIPvector used to produce them. The FRhK-4-Ssol-CTE#3 cell clone enabled the highest concentrations of the Ssol protein to be obtained in the supernatants collected after 72 hours of culture. This clone was submitted to a second series of 5 transduction cycles and the selection process repeated to obtain second generation clones. The most productive second generation clone (FRhK-4-Ssol-CTE#30) was amplified and used to produce greater quantities of supernatant. Subsequently, using a capture ELISA test, using a range of purified Ssol as protein marker, it was possible to estimate that the Ssol protein was secreted into the supernatant of the FRhK-4-Ssol-CTE#30 clone at concentrations varying from 5-10 μg/ml. The optimum conditions for producing the Ssol protein were determined experimentally by acting on the cell density parameters, serum concentration, culture temperature and secretion duration.

### Production and Purification of the Recombinant Ssol Protein

For large-scale production of Ssol protein, lots of 1.5-2.10⁸ sub-confluent cells of the FRhK-4-Ssol-CTE#30 clone were incubated at 35° C. for 4 days in 1 litre of DMEM-based culture media containing 0.5% of foetal calf serum. The supernatant containing the secreted Ssol protein was concentrated on an ultrafiltration unit and purified by affinity chromatography on an anti-FLAG antibody column. The material fixed to the column was eluted under non denaturing conditions by competition with the Flag peptide, and then separated by gel filtration to eliminate the Flag peptide and the low molecular weight contaminants.

The purified material was analysed by SDS-PAGE and silver nitrate staining. An intense, diffuse band, characteristic of glycoproteins was displayed with the expected size for the Ssol polypeptide (180-200 kDa). Analysis by Western blotting after SDS-PAGE using a specific rabbit polyclonal antibody of the S protein confirmed that the purified protein clearly corresponds to the ectodomain of the S protein. The degree of purity of the purified protein was estimated after SDS-PAGE and staining with ruby SYPRO. The quantification of fluorescence signals indicated that more than 90% of proteins eluted from the gel filtration were from the Ssol protein. The purified Ssol protein was next quantified with the help of a kit using the Bi-cinchoninic acid assay (BCA). After analysis of 3 independent productions, it was possible to obtain from 1.3-2.5 mg of Ssol protein per litre of culture supernatant. The overall purification yield, including all the stages (concentration, affinity purification and gel filtration) varies from 26-53%. The purified Ssol protein was then further characterised by N-terminal sequencing, mass spectrography and analytical ultra-centrifuging. From this it was determined that the purified Ssol protein is a soluble monomer of 182 kDa corresponding to the entire ectodomain of the S protein, but missing the signal peptide (amino acids 1-13).

### Preparation of an Oil-In-Water Adjuvant

The oil-in-water emulsion used in the subsequent examples is composed of an organic phase made of two oils (alpha-tocopherol and squalene), and an aqueous phase of phosphate buffered saline (PBS) containing polyoxyethylene sorbitan monooleate (Tween 80™) as emulsifying agent. Unless otherwise stated, the oil-in-water emulsion adjuvant formulations used in the subsequent examples were made comprising the following oil-in-water emulsion component (final concentrations given): 2.5% squalene (v/v), 2.5% alpha-tocopherol (v/v), 0.9% polyoxyethylene sorbitan monooleate (v/v), see WO 95/17210. This emulsion, termed GSK2 in the subsequent examples, was prepared as follows as a two-fold concentrate.

The emulsion is made by mixing under strong agitation an oil phase composed of hydrophobic components (α-tocopherol and squalene) and an aqueous phase containing the water soluble components (polyoxyethylene sorbitan monooleate and PBS mod (modified), pH 6.8). While stirring, the oil phase (1/10 total volume) is transferred to the aqueous phase (9/10 total volume), and the mixture is stirred for 15 minutes at room temperature. The resulting mixture is then subjected to shear, impact and cavitation forces in the interaction chamber of a microfluidizer (15000 PSI-8 cycles) to produce submicron droplets (distribution between 100 and 200 nm). The resulting pH is between 6.8±0.1. The emulsion is then sterilised by filtration through a 0.22 μm membrane and the sterile bulk emulsion is stored refrigerated in Cupac containers at 2 to 8° C. Sterile inert gas (nitrogen or argon) is flushed into the dead volume of the emulsion final bulk container for at least 15 seconds.

### Testing Adjuvanted Vaccine in a Mouse Model

BALB/c young adult mice (8 per group) received two injections, at 3 week intervals, into muscular tissue, of 2 μg of Ssol protein either without adjuvant, or with 50 μg of ═μL or 504 of the oil-in-water emulsion adjuvant (GSK2 adjuvant). These doses of adjuvants are traditionally used with small rodents and correspond to 1/10th of doses used in human medicine. Two groups of mice were associated with this research as controls, each being immunised with only one of the adjuvants. The mice sera were collected 3 weeks after each injection, and the specific humoral response of the SARS-CoV evaluated by anti-SARS ELISA, seroneutralisation and isotype analysis.

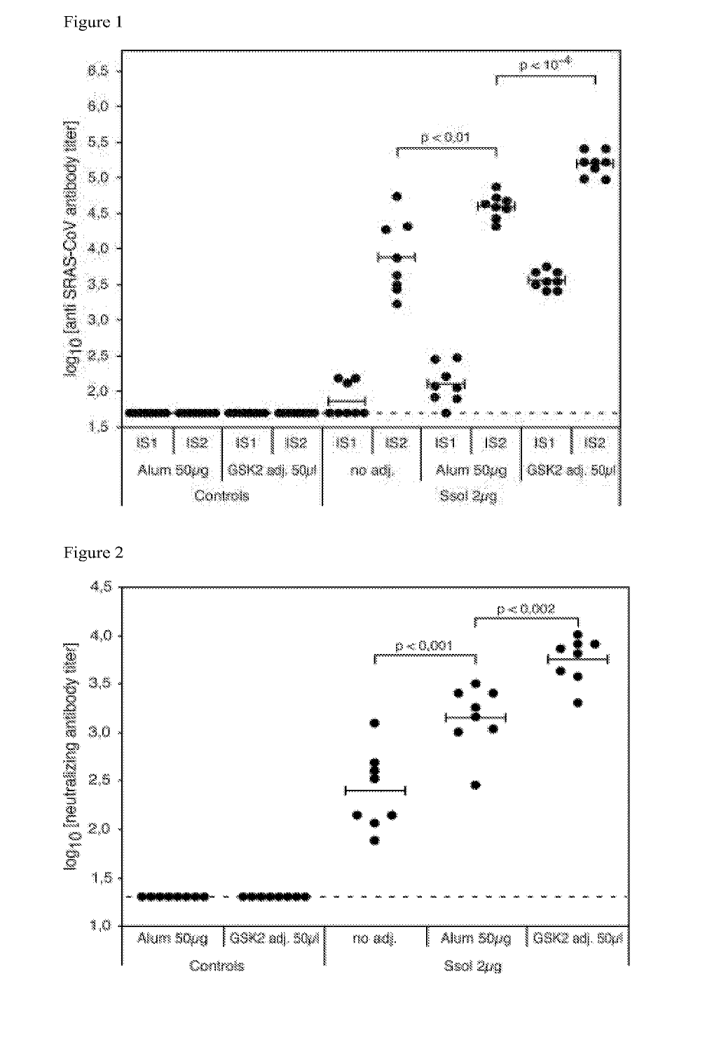

By ELISA (FIG. 1), the titers in antibodies of sera from control groups constantly remained below the limit of detection (1.7 log 10). After only one injection, the responses in antibodies induced by the protein with no adjuvant or with Alum adjuvant are weak (average titers of 1.9±0.2 log 10 and 2.1±0.3 log 10, respectively), confirming the results obtained previously. Contrariwise, the antibody titers induced by the protein with the oil-in-water emulsion adjuvant (GSK2 adjuvant) are very large from the first injection (average titer of 3.6±0.2 log 10). After two injections, a marked increase in titers in all the groups immunised with the protein is noted. The weakest response and the most heterogeneous one is observed when no adjuvant was used (average titer of 3.9±0.5 log 10). The adding of Alum to the immunogenic preparation enables the antibody response to be improved (average titer of 4.6±0.2 log 10; p<0.01). Tallying with the results observed after the first injection, the oil-in-water emulsion adjuvant (GSK2 adjuvant) markedly improved the immunogenicity of the Ssol protein after two injections, and the antibody titers obtained (average titers of 5.2±0.2 log 10) are significantly higher than those induced by the protein with Alum adjuvant (p<10⁻⁴).

The quality of the humoral response by the various immunogens was studied on the sera collected 3 weeks after the second injection. The neutralising antibody titers (FIG. 2) follow the hierarchy observed at the time of the analysis by ELISA. The weakest titers are obtained with the protein with no adjuvant (average titer of 2.3±0.4 log 10). The neutralising response is significantly improved by the addition of Alum (average titer of 3.1±0.3 log 10; p<0.001). Likewise, the addition to the protein of the oil-in-water emulsion adjuvant (GSK2 adjuvant) enables very large neutralising antibody titers to be achieved (average titers of 3.7±0.2 log 10), and significantly four-fold higher than those induced by the protein with Alum adjuvant (p<0.002).

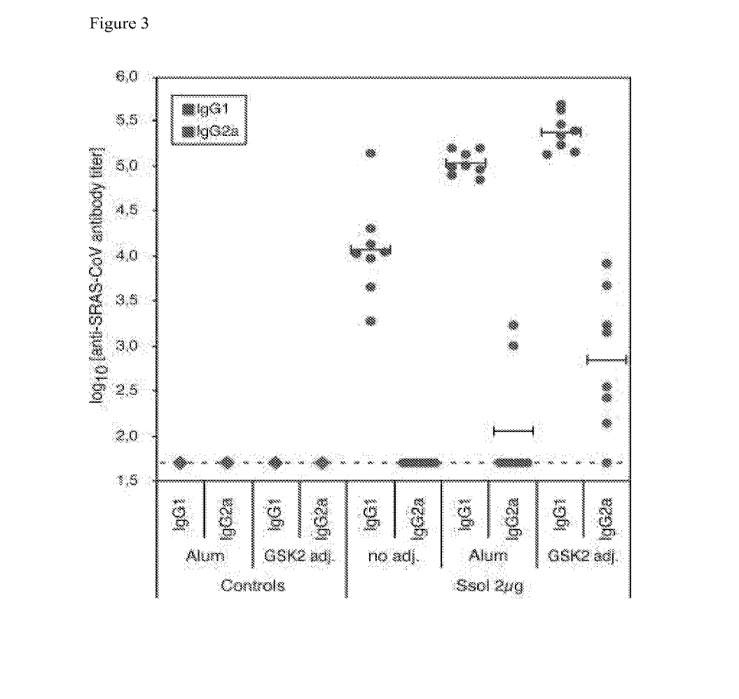

The specific IgG1 and IgG2a isotype titers to the SARS-CoV antigens were evaluated for each group by anti-SARS ELISA on the sera collected 3 weeks after the last injection (FIG. 3). The immunisations with the protein with no adjuvant or with the protein with Alum adjuvant almost exclusively induce IgG1s. The addition to the Ssol protein of the oil-in-water emulsion adjuvant (GSK2 adjuvant) enables the induction of even higher IgG1 titers (average titer 5.4±0.2 log 10) as well as IgG2a titers to higher levels than in the presence of alum (average titers 2.8±0.7 versus 2.1±0.6; p<0.05). The average ratio of IgG1 over IgG2a is 840 in the presence of GSK2 adjuvant and more than 1600 in the presence of alum. These results demonstrate that the immune responses induced by the protein with no adjuvant or with Alum are predominantly type 2. The addition of the oil-in-water emulsion adjuvant (GSK2 adjuvant) to the Ssol protein although in favour of a TH-2 type immune response enables the induction of a weak albeit heterogeneous TH1-type immune response.

### Example 2

### Testing Adjuvanted Vaccine in a Hamster Model

Syrian golden hamsters (6 per group) were injected twice, at 3-week intervals, into muscular tissue, with 2 μg or 0.2 μg of Ssol protein either with 50 μg of Alum or 50 μL of the oil-in-water emulsion adjuvant (GSK2 adjuvant). These doses of adjuvants are traditionally used with small rodents and correspond to the 1/10th doses used in human medicine. Two groups of hamsters were associated with this experiment as controls, each being immunised with only one of the adjuvants. Another group of hamsters was injected with 2 μg (S-equivalent) of purified and β-propiolactone-inactivated SARS-CoV virions (BPL-SCoV) with 50 μg of Alum, which constitutes a potential vaccine against SARS. The hamster sera were collected 3 weeks after each injection (IS1 and IS2, respectively) and 3 months after the second injection (IS2bis), and the specific humoral response of the SARS-CoV evaluated by anti-SARS ELISA and seroneutralisation analysis.

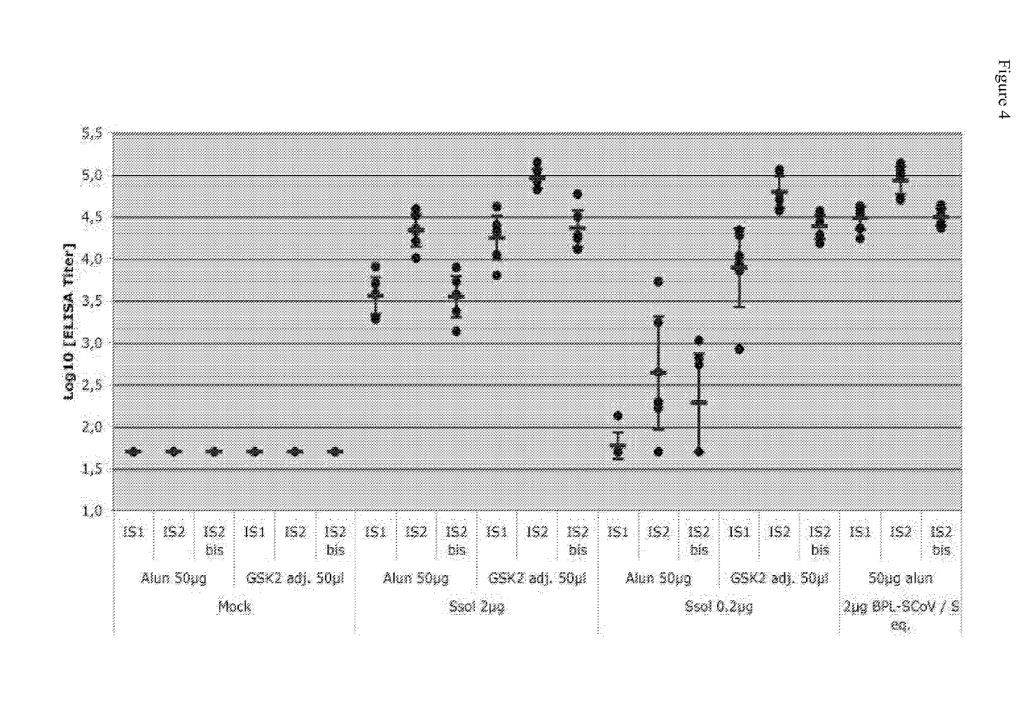

By ELISA (FIG. 4), the titers in antibodies of sera from control groups constantly remained below the limit of detection (1.7 log 10). At the 2 μg Ssol dose, the oil-in-water emulsion adjuvant (GSK2 adjuvant) markedly improved the immunogenicity of the Ssol protein after one (IS1) and two (IS2) injections, and the antibody titers obtained (average titers of 4.2±0.3 log 10 and 5.0±0.1 log 10) are 0.6 and 0.7 log 10 higher respectively than those induced by the protein with Alum adjuvant (p<10⁻³).

After only one injection of 0.2 μg of Ssol, the response observed when Alum adjuvant was used is weak and close to the limit of detection (average titers of 1.8±0.2 log 10). Contrariwise, the antibody titers induced by the protein with the oil-in-water emulsion adjuvant (GSK2 adjuvant) are high from the first injection (average titer of 3.9±0.5 log 10) reaching levels higher, albeit more heterogeneous, than those achieved after a single injection of 2 μg Ssol in the presence of alum. After two injections, a marked increase in titers in both groups immunised with the protein is noted. The weakest response and the most heterogeneous one is observed when the Alum adjuvant was used (average titer of 2.6±0.7 log 10). The addition of the oil-in-water emulsion adjuvant (GSK2 adjuvant) to the immunogenic preparation enables the antibody response to be strongly improved (average titer of 4.8±0.2 log 10; p<10⁻⁴).

Remarkably, after the second injection, 0.2 μg and 2 μg Ssol with the oil-in-water emulsion adjuvant (GSK2 adjuvant) achieved comparable high-titer responses in all immunized hamsters (4.8±0.2 log 10 versus 5.0±0.1 log 10 titer). This indicates that the use of the oil-in-water emulsion adjuvant (GSK2 adjuvant) could enable dose-sparing vaccine strategies against SARS.

The quality of the humoral response induced by 0.2 μg Ssol or 2 μg (S-equivalent) inactivated virions was studied on the sera collected 3 months after the second injection. The neutralising antibody titers (FIG. 5) follow the hierarchy observed at the time of the analysis by ELISA. The titers obtained with the protein with Alum remained below the limit of detection (1.3 log 10). The neutralising response is strongly improved by the addition of the oil-in-water emulsion adjuvant (GSK2 adjuvant) (average titer of 2.7±0.2 log 10; p<10⁻⁷). This response was clearly similar to the response induced by 2 μg (S-equivalent) inactivated virions (average titer of 2.5±0.2 log 10).

### Challenge Infection of Ssol-Immunized Hamsters

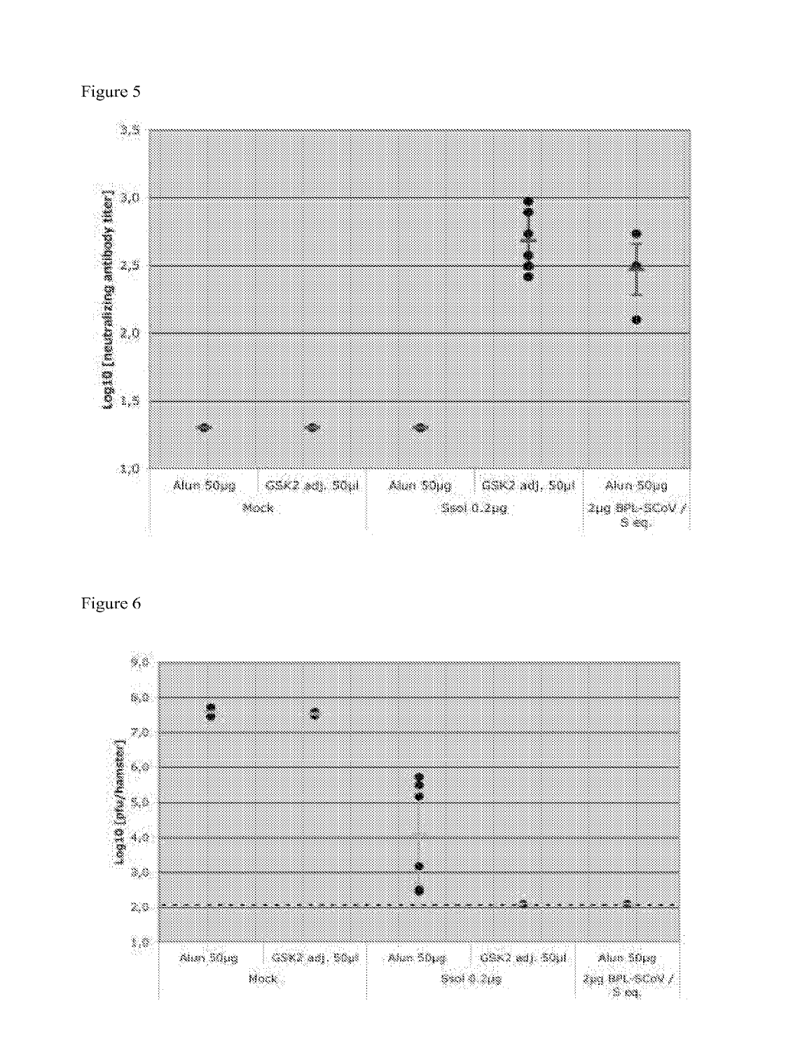

At 3 months post-immunization, selected groups of hamsters were challenged by intranasal inoculation of 10⁵ pfu of SARS-CoV and euthanized 4 days later in order to assess viral replication. Viral loads were evaluated in the lungs (FIG. 6) and in the upper respiratory tract (URT), i.e. pharynx plus trachea (FIG. 7) of each animal. We observed a robust and consistent replication of the virus in the lungs of each mock-immunized animal (7.5±0.1 log 10 pfu). In addition, viral replication was documented in the upper respiratory tract of mock-vaccinated hamsters (5.0±0.3 log 10 pfu). SARS-CoV loads remained detectable both in lungs (4.1±1.4 log 10 pfu) and URT (2.5±0.4 log 10 pfu) of animals immunized with 0.2 μg Ssol with Alum. In sharp contrast, in spite of robust virus replication in this animal model, no infectious virus was detectable in any of the hamsters immunized with Ssol and the oil-in-water emulsion adjuvant (GSK2 adjuvant) (log 10 pfu/organ<2.1). These data provide evidence for a more than 10²-fold reduction of SARS-CoV replication in the lungs of hamsters immunized with Ssol and the oil-in-water emulsion adjuvant (GSK2 adjuvant) compared to hamsters immunized with Ssol and Alum. This high level of protection achieved with Ssol and the oil-in-water emulsion adjuvant (GSK2 adjuvant) is comparable to that observed in hamsters immunized with inactivated virions and Alum.

Interestingly, a single injection of 2 μg Ssol with the oil-in-water emulsion adjuvant (GSK2 adjuvant) induced similar high ELISA titers of anti-SARS antibodies (4.2±0.3 log 10 pfu) as 2 injections of 0.2 μg Ssol with the oil-in-water emulsion adjuvant (GSK2 adjuvant) at the time of challenge (4.4±0.1 log 10 pfu) (FIG. 4). Given the fact that this latter vaccination schedule protects immunized hamsters against SARS challenge (FIGS. 6 and 7), it can be anticipated that a single injection of 2 μg Ssol with the oil-in-water emulsion adjuvant (GSK2 adjuvant) will also induce a protective response. This indicates that the use of the oil-in-water emulsion adjuvant (GSK2 adjuvant) could enable single-dose vaccination strategies against SARS.

### Histopathological Analysis of the Lungs of Challenged Hamsters

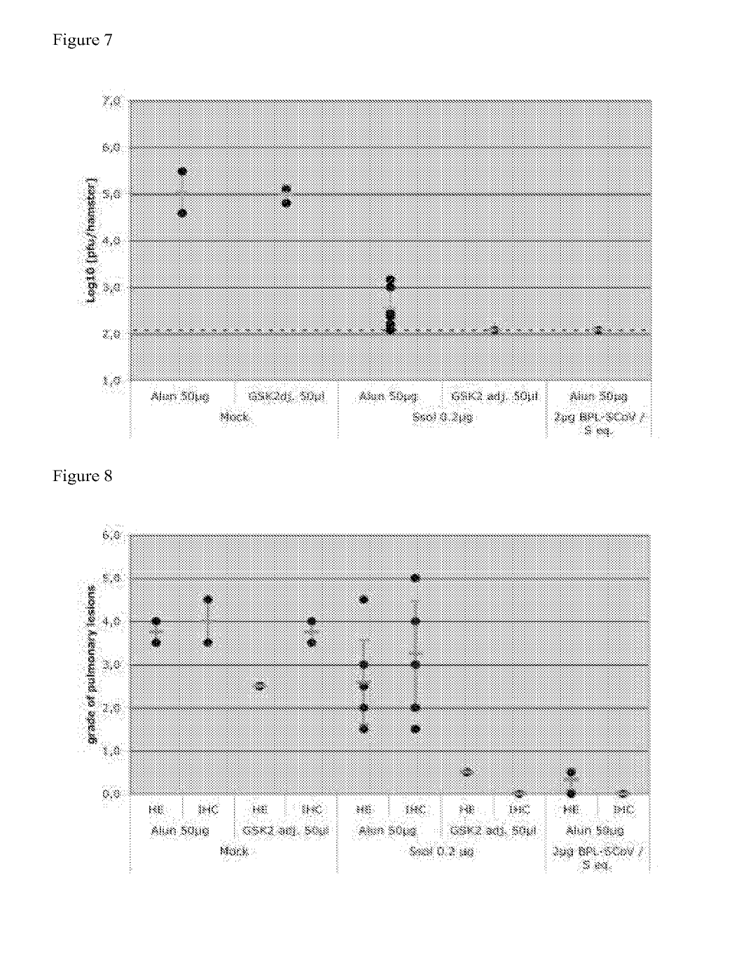

After challenge, the lungs of hamsters immunized with 0.2 μg of Ssol protein were subjected to histopathological examination using Hemalun-Eosin stain and immunohistochemistry analysis using anti-SARS-CoV polyclonal antibody. FIG. 8 shows the scores of pulmonary inflammation and lesions (HE) and the scores of viral antigen loads (IHC) on a 1-10 scale. In mock vaccinated-animals, characteristic lesions of acute viral pneumonia were observed with diffuse lesions of exsudative alveolitis, diffuse condensation of the lung parenchyma and diffuse alveolar damage (score=3.1±0.6). Accordingly, viral antigens were detected within these foci of alveolitis and also in the epithelium of the trachea and the broncho-alveolar tree (score=3.9±0.4). Both lesion scores (2.6±1.0) and viral antigen loads (3.3±1.2) remained high in lungs of animals immunized with 0.2 μg Ssol with Alum. In sharp contrast, in Ssol and the oil-in-water emulsion adjuvant (GSK2 adjuvant)-vaccinated hamsters, no specific lesion of alveolitis or pneumonia was detected in the lungs (score=0.5±0.0) and viral antigens were not detected in any of the animals despite extensive IHC screening of respiratory tract sections from the upper respiratory tract (pharynx-trachea, data not shown) and lungs (FIG. 8).

These results confirm that hamsters vaccinated with Ssol and the oil-in-water emulsion adjuvant (GSK2 adjuvant) were fully protected from SARS-CoV challenge, as indicated by the absence of detectable viral antigen in the upper and lower repiratory tracts, and the absence of pneumonitis.

Long Term Protection from Challenge Infection of Ssol-Immunized Hamsters

Long term protection was studied for the group of hamsters immunized twice with 2 μg of Ssol. Eight months after the second injection, the neutralising antibody response was improved by the addition of the oil-in-water emulsion adjuvant (GSK2 adjuvant) (average titer of 2.6±0.2 log 10; p<0.005) when compared to the addition of alum (average titer 1.8±0.4 log 10) (FIG. 21). This response was clearly similar to the response induced by 2 μg (S-equivalent) inactivated virions (average titer of 2.6±0.2 log 10).

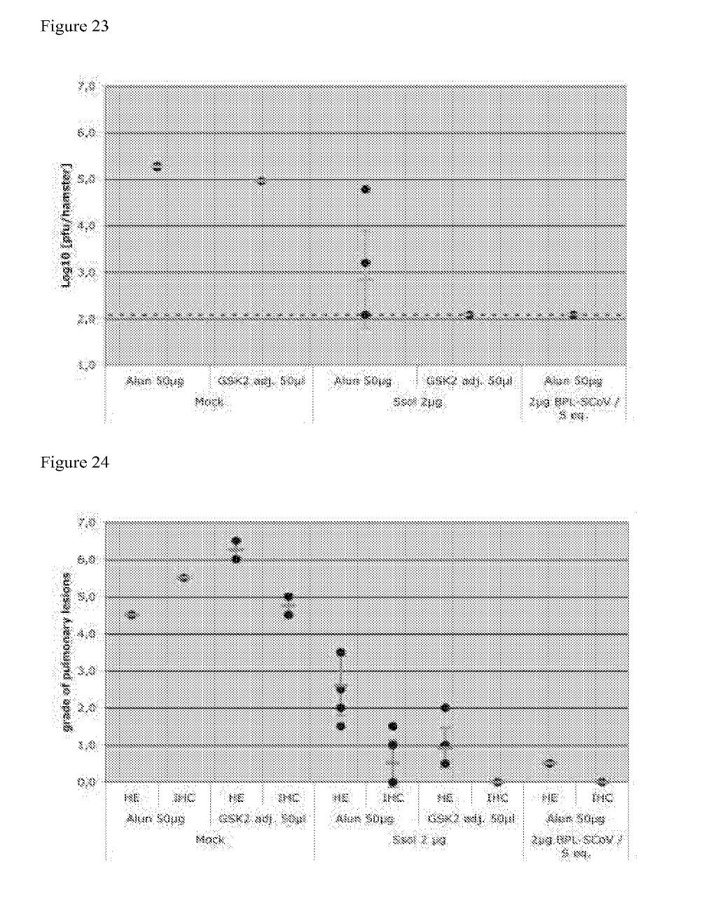

The hamsters were then challenged by intranasal inoculation of 10⁵ pfu of SARS-CoV and euthanized 4 days later in order to assess viral replication. Viral loads were evaluated in the lungs (FIG. 22) and in the upper respiratory tract (URT) (FIG. 23) of each animal. Consistent with the results described above, a robust virus replication was observed in both the lungs and URT of mock-vaccinated animals (7.7±0.2 log 10 pfu and 5.1±0.2 log 10 pfu in the lungs and URT, respectively). In animals immunized with 2 μg Ssol with Alum, SARS-CoV loads were detectable in both the lungs and URT in 2 out of 5 animals and a high viral load (4.8 log 10 pfu) was observed in the URT in one animal. In sharp contrast, no infectious virus was detectable in any of the hamsters immunized with Ssol and the oil-in-water emulsion adjuvant (GSK2 adjuvant) (log 10 pfu/organ<2.1). This high level of protection achieved with Ssol and the oil-in-water emulsion adjuvant (GSK2 adjuvant) is comparable to that observed in hamsters immunized with inactivated virions and Alum.

In addition, following challenge and euthanasia, the lungs of the hamsters were subjected to histopathological examination using Hemalun-Eosin stain (HE) and immunohistochemistry (IHC) analysis using anti-SARS-CoV polyclonal antibody (FIG. 24). As described above, in mock vaccinated-animals, characteristic lesions of acute viral pneumonia were observed with diffuse lesions of exsudative alveolitis, diffuse condensation of the lung parenchyma and diffuse alveolar damage (score=5.4±0.9) and viral antigens were detected (score 5.1±0.4). In the lungs of animals vaccinated with 2 μg of Ssol and the oil-in-water emulsion adjuvant (GSK2 adjuvant), lesion scores were significantly reduced (0.9±0.5, p<10⁻⁴) and viral antigen loads were undetectable whereas in animals vaccinated with 2 μg of Ssol with Alum, a more modest reduction of lesion scores was observed (score=2.6±0.8) and viral antigen remained detectable in 2 out of 5 animals (score=0.5±0.6).

Altogether, these data provide evidence for the potential for long term protection by the Ssol protein and the oil-in-water emulsion adjuvant (GSK2 adjuvant).

Protection of Hamsters from Challenge Infection with a Single Injection of Ssol

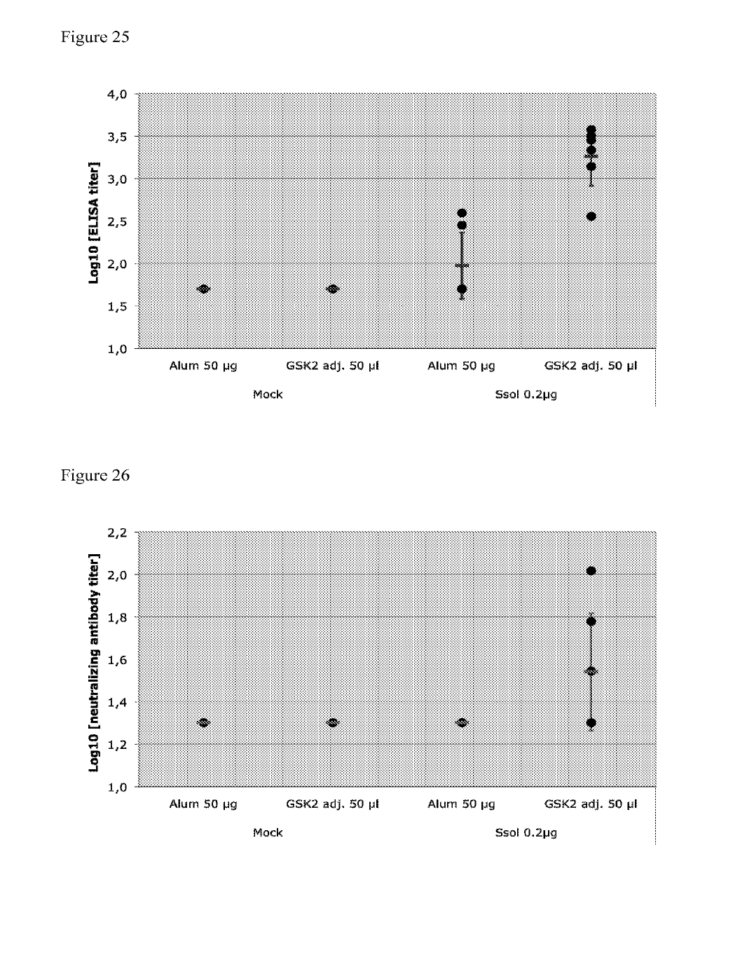

Protection after immunization with a single injection of Ssol was studied for a dose of 0.2 μg of Ssol injected into muscular tissue, either with 50 μg of Alum or 50 μL of the oil-in-water emulsion adjuvant (GSK2 adjuvant). Two weeks after the injection, the ELISA antibody response (FIG. 25) was strongly improved by the addition of the oil-in-water emulsion adjuvant (GSK2 adjuvant) (average ELISA titer of 3.3±0.3 log 10; p<0.001) when compared to the addition of alum (average titer 2.0±0.4 log 10). The neutralising antibody titers (FIG. 26) follow the hierarchy observed by the ELISA analysis. The titers obtained with the protein with Alum remained below the limit of detection (1.3 log 10) for each animal. The neutralising response is improved by the addition of the oil-in-water emulsion adjuvant (GSK2 adjuvant) and three out of 6 immunized hamsters had detectable antibody responses (average titer of 1.5±0.3 log 10; p<0.1).

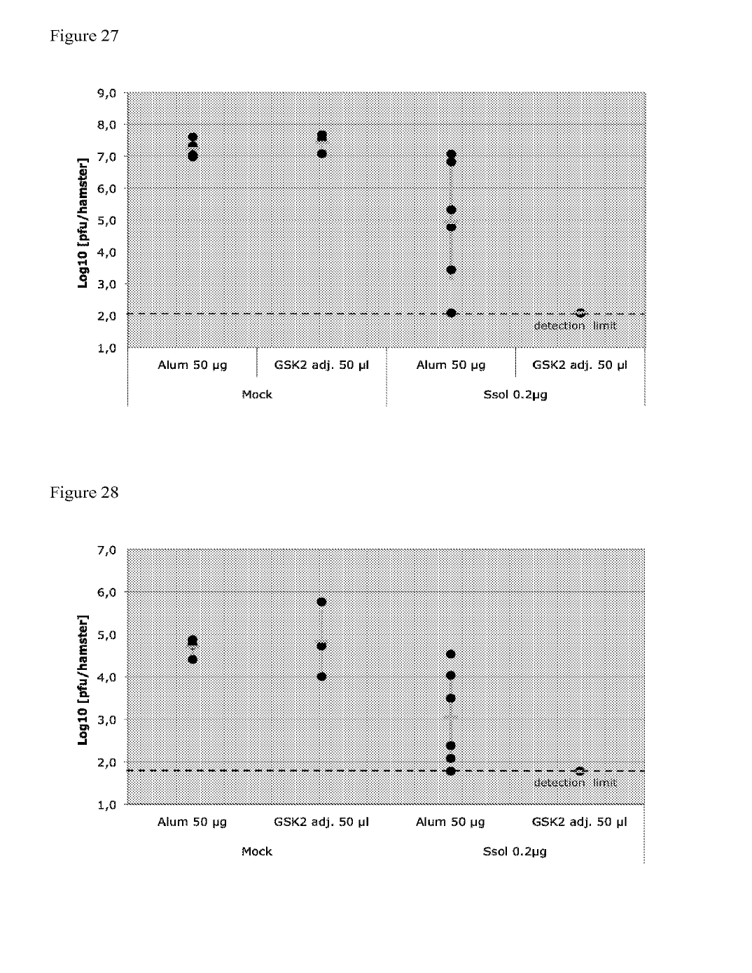

The hamsters were challenged three weeks after immunization by intranasal inoculation of 10⁵ pfu of SARS-CoV and euthanized 4 days thereafter in order to assess viral replication. Viral loads were evaluated in the lungs (FIG. 27) and in the upper respiratory tract (URT) (FIG. 28) of each animal. Consistent with the results described above, a robust virus replication was observed in both the lungs and URT of mock-vaccinated animals (7.3±0.3 log 10 pfu and 4.8±0.5 log 10 pfu in the lungs and URT, respectively). After immunization with 0.2 μg Ssol with Alum, moderate to high SARS-CoV loads were detectable in both the lungs and URT in 5 out of 6 animals (4.9±1.8 log 10 pfu and 3.0±1.0 log 10 pfu in the lungs and URT, respectively). In sharp contrast, no infectious virus was detectable in any of the hamsters immunized with Ssol and the oil-in-water emulsion adjuvant (GSK2 adjuvant) (log 10 pfu/lungs<2.1 and log 10 pfu/URT<1.8).

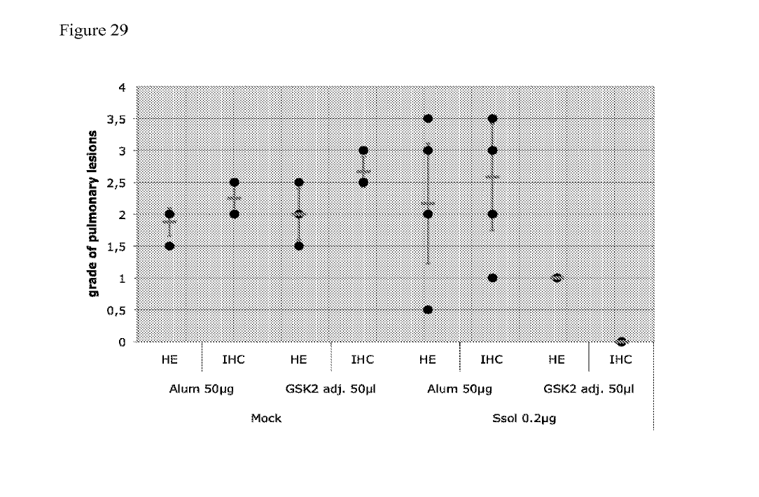

In addition, following challenge and euthanasia, the lungs of the hamsters were subjected to histopathological examination using Hemalun-Eosin stain (HE) and immunohistochemistry (IHC) analysis using anti-SARS-CoV polyclonal antibody (FIG. 29). As described above, in mock vaccinated-animals, characteristic lesions of acute viral pneumonia were observed with diffuse lesions of exsudative alveolitis, diffuse condensation of the lung parenchyma and diffuse alveolar damage (score=1.9±0.3) and viral antigens were detected (score 2.4±0.3). In the lungs of animals vaccinated with 0.2 μg of Ssol and the oil-in-water emulsion adjuvant (GSK2 adjuvant), lesion scores were significantly reduced (1.0±0.0, p<10⁻⁴) and viral antigen loads were undetectable whereas in animals vaccinated with 0.2 μg of Ssol with Alum, no reduction of lesion scores was observed (score=2.2±0.9) and viral antigen remained detectable in each of the 6 animals (score=2.6±0.8).

Altogether, these data provide evidence for the potential for high level protection induced by a single, low-dose injection of Ssol protein and the oil-in-water emulsion adjuvant (GSK2 adjuvant).

### Example 3

### A) Humoral Immune Response to Adjuvanted Ssol Protein in BALB/c Mice

Female BALB/c mice aged 6-8 weeks were obtained from Harlan Horst, The Netherlands. Mice (23 mice/group) were injected intramuscularly on days 0 and 21 with 2, 0.2 or 0.02 μg Ssol protein without adjuvant (“Plain”), adjuvanted with 50 μg Alum or with the oil-in-water emulsion adjuvant (GSK2 adjuvant). Three additional groups of mice were included as controls, each being immunised with PBS, Alum or the GSK2 adjuvant alone.

### Preparation of Non Adjuvanted Ssol Antigen

The formulations were prepared extemporaneously according to the following sequence: water for injection+Ssol antigen (quantities are added in order to reach final concentrations of 40 μg/ml or 4 μg/ml or 0.4 μg/ml), 5 min mixing on an orbital shaking table at room temperature+NaCl 1500 mM (in order to reach a final concentration of 150 mM), 5 min mixing on an orbital shaking table at room temperature. The injections occurred within an hour following the end of the formulation.

### Preparation of Alum-Adjuvanted Ssol

The vaccine preparation was made according the following sequence: water for injection+aluminium hydroxide (quantities are added in order to reach a final concentration of 1000 μg/ml)+Ssol antigen (in order to reach a final concentration of 40 μg/ml, 4 μg/ml or 0.4 μg/ml), 30 min mixing on an orbital shaking table at room temperature+NaCl 1500 mM (in order to reach a final concentration of 150 mM), 5 min mixing at room temperature on an orbital shaking table. The vaccine was prepared six days before the first immunization in the first study and kept at 4° C. until injection.

### Preparation of GSK2 Adj-Adjuvanted Ssol

The formulations were prepared extemporaneously according the following sequence: water for injection+10-fold concentrated phosphate buffered saline+Ssol antigen (quantities were added in order to reach final concentrations of 40 μg/ml or 4 μg/ml or 0.4 μg/ml), 5 min mixing on an orbital shaking table at room temperature, +2-fold concentrated GSK2 adjuvant, 5 min mixing on an orbital shaking table at room temperature. The injections occurred within two hours following the end of the formulation.

### Analysis of Humoral Response

The humoral response was evaluated on sera prepared from blood samples taken from individual mice (8 mice per group) at 14 days post-immunization (day 35 timepoint). Detection of the presence of anti-SARS-CoV specific antibodies and isotype analysis were performed by indirect ELISA using a lysate of VeroE6 cells infected by SARS-CoV as antigen or of non-infected VeroE6 cells as a negative control. Titers were calculated as the reciprocal of the dilution of serum giving an OD of 0.5 after revealing with polyclonal anti-mouse IgG(H+L) antibodies coupled to peroxydase (NA931V, Amersham) followed by addition of TMB and H2O2 (KPL). For the analysis of isotypes polyclonal sera specific for mouse IgG1 and IgG2a antibodies were used (Southern Biotech).

### Anti-SARS-CoV Antibodies.

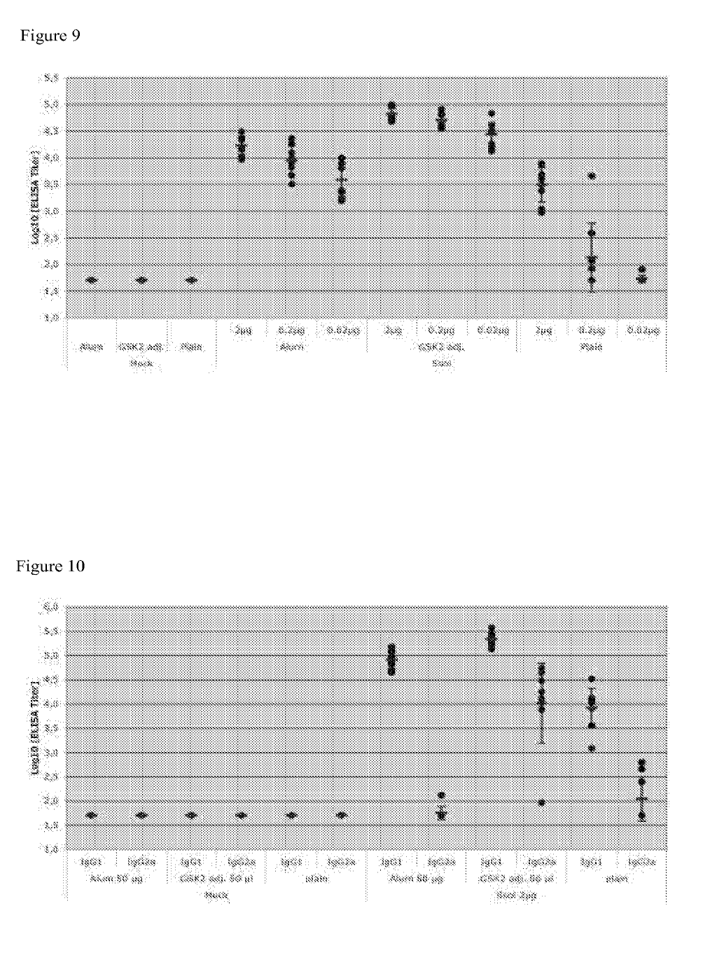

A dose-dependent anti-SARS-CoV antibody response was observed in mice immunized with the Ssol protein either without adjuvant or in the presence of Alum or of the oil-in-water emulsion adjuvant (GSK2 adjuvant) (FIG. 9). The antibody response was found to be significantly higher in mice immunized with Ssol in the presence of adjuvant as compared to mice immunized with non-adjuvanted Ssol. The response was significantly higher for mice immunized with the oil-in-water emulsion adjuvant (GSK2 adjuvant)-adjuvanted Ssol protein as compared to mice immunized with Alum-adjuvanted Ssol (p<10⁻⁴); antibody titers induced with the lowest dose of Ssol (0.02 μg) in the presence of the oil-in-water emulsion adjuvant (GSK2 adjuvant) were found superior to those induced with the highest dose of Ssol (2 μg) in the presence of alum (p=0.08).

### Isotype Analysis of Anti-SARS-CoV Antibodies.

An isotype analysis for IgG1 and IgG2a antibodies specific of SARS-CoV was performed by ELISA on sera prepared from the “Plain”, Alum-adjuvanted and the oil-in-water emulsion adjuvant (GSK2 adjuvant)-adjuvanted groups immunized at a dose of 2 μg Ssol (8 mice per group). Results are shown in FIG. 10.

In mice immunized either with the non-adjuvanted Ssol protein or with the Ssol protein adjuvanted with alum, the response was found to be strongly biased towards the IgG1 isotype whereas very low levels of IgG2a antibodies were detected. In mice immunized with the oil-in-water emulsion adjuvant (GSK2 adjuvant)-adjuvanted Ssol protein, high titers of IgG1 (5.3±0.1 log 10 titers) antibodies were reached. Interestingly, titers of IgG2a (4.0±0.8 log 10 titers) antibodies were significantly increased except in one animal as compared to mice immunized with the alum-adjuvanted Ssol protein (p<10⁻⁵).

### Neutralizing Antibodies

The presence of neutralizing antibodies was determined by a standard seroneutralization assay on FRhK-4 cells using 100 TCID50 of SARS-CoV per well. Serial two-fold dilutions of heat inactivated sera (56° C. for 30 min) were used from dilution 1:20 on and tested in duplicate. Neutralizing titers were determined according to the method of Reed and Munsch (Am J Hyg 1938; 27:493-97) as the reciprocal of the dilution that neutralizes virus infectivity in 50% of the wells (2 out of 4 wells).

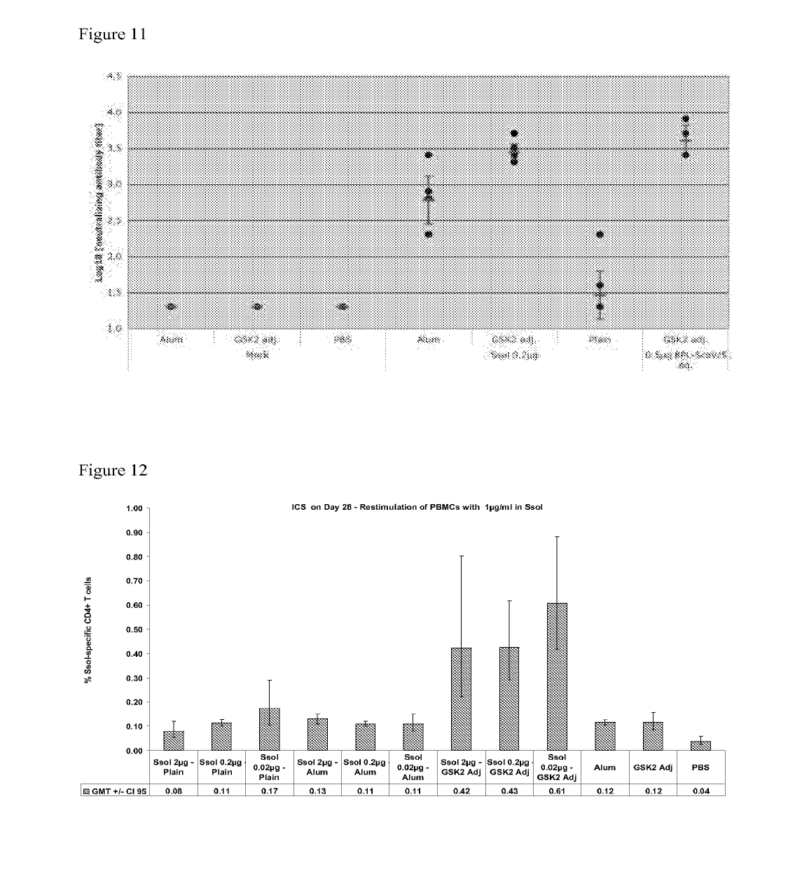

Sera prepared 14 days post-immunization from individual mice immunized with 0.2 μg of Ssol either without or in the presence of alum or of the oil-in-water emulsion adjuvant (GSK2 adjuvant) (8 mice/group) were analyzed for the presence of antibodies neutralizing SARS-CoV. Sera from mice immunized with an inactivated whole virus preparation at a dose equivalent to 0.5 μg S protein in the presence of the oil-in-water emulsion adjuvant (GSK2 adjuvant) were included for comparison. Results are shown in FIG. 11.

In mice immunized with the oil-in-water emulsion adjuvant (GSK2 adjuvant)-adjuvanted Ssol protein, neutralizing antibody titers (3.4±0.1 log 10 titers) were 0.6 log 10 higher than in mice immunized with the alum-adjuvanted Ssol protein (2.8±0.3 log 10 titers, p<0.001) whereas in mice immunized with the non-adjuvanted Ssol protein neutralizing titers remained undetectable for 6 out of 8 mice (<1.3 log 10 titers). Noticeably, in the presence of the oil-in-water emulsion adjuvant (GSK2 adjuvant), neutralizing antibody titers were comparable with 0.2 μg of Ssol protein as compared to 0.5 μg S-equivalent whole virus antigen (3.6±0.2 log 10 titers).

### B) Cellular Immune Response to Adjuvanted Ssol Protein in BALB/c Mice

The cell-mediated immune responses of the “Plain”, Alum-adjuvanted and GSK2 adj-adjuvanted groups of BALB/c mice were investigated, as summarised in Table A below.

TABLE A

| | Timepoint | | |
|---|---|---|---|
| | (days after | Sample | |
| Read-out | 1st injection) | type | Analysis method |
| CD4, CD8, | D 28 | PBMC | Intracellular cytokine |
| IL-2, IFN-γ | | | staining (ICS) |
| | | | (FACS analysis) |
| CD4, CD8, | D 35 | Spleen | ICS (FACS analysis) |
| IL-2, IFN-γ | | | |
| IL-5, IL-13 | D 35 | Spleen | Cytometric Bead Array (CBA) |
| and IFN-γ | | | (FACS analysis) |

Cellular responses were measured on PBMC and spleens from 15 mice/group. PBMC were harvested 7 days post-immunization and spleens were harvested 14 days post-immunization. PBMC were tested on 5 pools of 3 mice and spleens were tested on 4 pools of 2 mice per group.

### Intracellular Cytokine Staining (ICS)

After lysis of red blood cells with a lysis buffer (BD pharmingen), in vitro antigen stimulation of PBMC was carried out at a final concentration of 10⁷ cells/ml (microplate 96 wells) with a concentration of Ssol at 1 μg/ml final, and then incubated 2 hours at 37° C. with the addition of anti-CD28 and anti-CD49d (1 μg/ml for both). Following the antigen restimulation step, cells were incubated overnight in presence of Brefeldin (1 μg/ml) at 37° C. to inhibit cytokine secretion.

Spleens were collected from mice and pooled (4 pools of 2 mice/group) in medium RPMI+Add. RPMI+Add-diluted PBL suspensions were adjusted to 10⁷ cells/ml in RPMI 5% fetal calf serum. In vitro antigen stimulation of spleen cells was carried out with Ssol 1 μg/ml final and then incubated 2 hrs at 37° C. with the addition of anti-CD28 and anti-CD49d (1 μg/ml for both). Following the antigen restimulation step, cells were incubated overnight in presence of Brefeldin (1 μg/ml) at 37° C. to inhibit cytokine secretion.

After overnight at 4° c., cell staining was performed as follows: cell suspensions were washed, resuspended in 50 μl of PBS 1% FCS containing 2% Fc blocking reagent (1/50; 2.4G2). After 10 minutes incubation at 4° C., 50 μl of a mixture of anti-CD4-PE (1/50) and anti-CD8a perCp (1/50) was added and incubated 30 minutes at 4° C. After a washing in PBS 1% FCS, cells were permeabilized by resuspending in 200 μl of Cytofix-Cytoperm (Kit BD) and incubated 20 min at 4° C.

Cells were then washed with Perm Wash (Kit BD) and resuspended with 50 μl of a anti-IFNγ-APC (1/50)+anti-IL-2 FITC (1/50) diluted in PermWash. After 2 hours incubation at 4° C., cells were washed with Perm Wash and resuspended in PBS 1% FCS+1% paraformaldehyde. Sample analysis was performed by FACS. Live cells were gated (FSC/SSC) and acquisition was performed on ˜20,000 events (lymphocytes CD4). The percentages of IFNγ+ or IL2+ were calculated on CD4+ gated populations.

### Dosage of Cytokine Secretion (CBA)

Dosage of cytokines in restimulation supernatant was also performed on PBMC from spleen 14 days after the immunization. Spleen was collected from mice and pooled (4 pools of 2 mice/group) in medium RPMI+Add. PBMC suspensions were adjusted to 10⁷ cells/ml in RPMI 5% fetal calf serum. In vitro antigen stimulation of PBMC was carried out with Ssol 1 μg/ml final and then incubated 72 hrs at 37° C. The supernatant was harvested and stored at −70° c. until testing by CBA (Cyokine Bead Assay)-flex (BD Kit) for IFNγ, IL-5 and IL-13 detection.

Bead populations with distinct fluorescence intensities were coated with capture antibodies specific for IFN-γ, IL5 and IL-13 proteins. Bead populations were mixed together to form the cytometric bead array (CBA) that was resolved in the FL3 channel of a BD FACS brand flow cytometer. The cytokine capture beads were mixed with the PE-conjugated detection antibodies and then incubated with recombinant standards or test samples to form sandwich complexes. Following acquisition of the sample data using the flow cytometer, the sample results were generated in graphical and tabular format.

Mouse cytokine standards were reconstituted and diluted by serial dilutions using the assay diluent. Mouse cytokine capture bead suspensions were pooled, mixed and transferred to each assay tube (50 μl/tube). Standard dilutions and test samples were added to the appropriate sample tubes (50 μl/tube) followed by 50 μl of PE detection reagent. All samples and standards were incubated for 2 hours at room temperature in the dark. After the incubation, all reaction tubes were washed with 1 ml of wash buffer, and centrifuged at 200×g for 5 minutes. After decanting, standards and samples were resuspended in 300 μl of wash buffer. Standards and samples were read on a FACSCalibur flow cytometer with BD FACSComp software for setting up the cytometer and CellQuest_software for the analysis of the samples, followed by a subsequent analysis (calculation of sample concentrations with standard curve) using the BD CBA software.

### CD4+ T Cell Responses in PBMC

At each antigen dose, significantly higher (p<0.05) CD4+ T cell responses were induced in mice immunized with Ssol protein adjuvanted with GSK2 adjuvant compared to mice immunized with Alum-adjuvanted Ssol or the non-adjuvanted Ssol protein (FIG. 12). Alum-adjuvanted Ssol or the non-adjuvanted antigen induced a similar level of CD4+ T cell responses as achieved by immunization with adjuvants alone or PBS. Similar levels of CD4+ T cell responses was observed after immunization of mice with 2 μg, 0.2 μg or 0.02 μg Ssol protein adjuvanted with the GSK2 adjuvant (FIG. 12).

### CD4+ T Cell Responses in Spleen

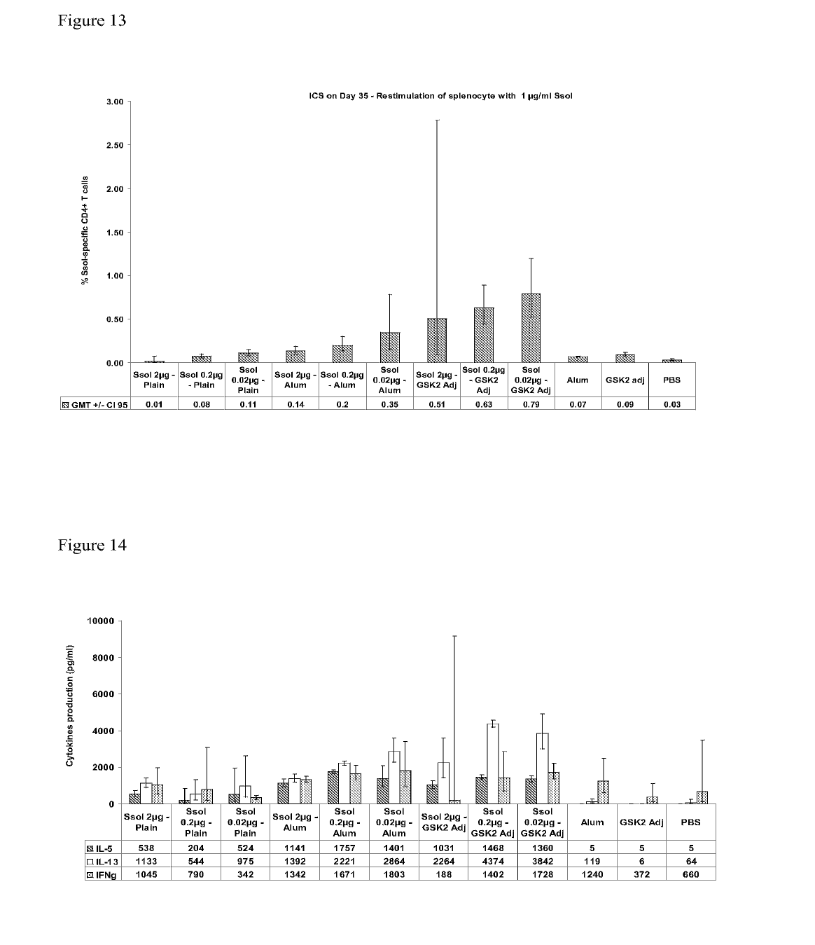

At each antigen dose, higher CD4+ T cell responses were induced in mice immunized with Ssol protein adjuvanted with GSK2 adjuvant compared to mice immunized with Alum-adjuvanted Ssol or non-adjuvanted Ssol protein (FIG. 13). Alum-adjuvanted Ssol or the non-adjuvanted antigen induced a similar level of CD4+ T cell responses as achieved by immunization with adjuvants alone or PBS. Similar levels of CD4+ T cell responses was observed after immunization of mice with 2 μg, 0.2 μg or 0.02 μg Ssol protein adjuvanted with the GSK2 adjuvant (FIG. 13).

Cytokine Secretion from Spleen Cells.

Higher levels of IL-5, IL-13 and IFN-γ cytokines were induced in mice immunized with Alum-adjuvanted Ssol or Ssol protein adjuvanted with GSK2 adjuvant compared to mice immunized with the non-adjuvanted Ssol protein (FIG. 14). Both adjuvants (Alum and GSK2 adjuvant), induced a mixed Th1-type (IFN-γ) and Th2-type (IL-5 and IL-13) cytokine profiles.

### Example 4

### A) Humoral Immune Responses to Adjuvanted Ssol Protein in C57B1/6 Mice

The same experimental protocol as described in Example 3 for BALB/c mice was carried out on female C57B1/6 mice aged 6-8 weeks obtained from Harlan Horst, The Netherlands. For the analysis of isotypes polyclonal sera specific for mouse IgG1 and IgG2b antibodies were used (Southern Biotech).

### Anti-SARS-CoV Antibodies

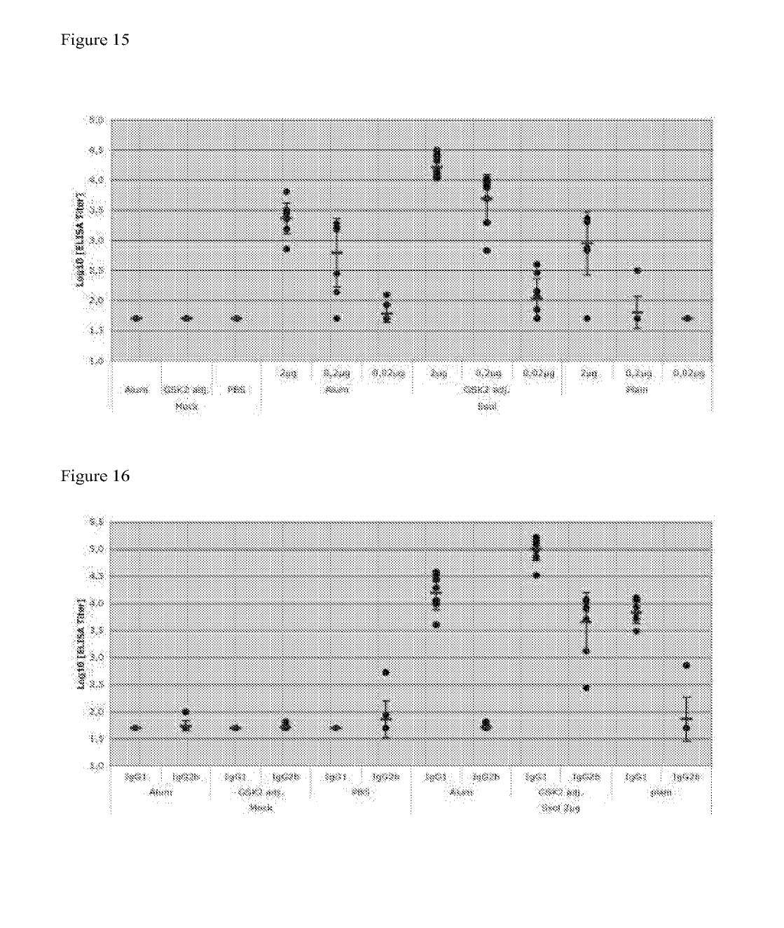

A dose-dependent anti-SARS-CoV antibody response was observed in mice immunized with the Ssol protein either without adjuvant or in the presence of Alum or of the oil-in-water emulsion adjuvant (GSK2 adjuvant) (FIG. 15). At each antigen dose, the antibody response was found to be significantly (0.3-1.9 log 10) higher in mice immunized with Ssol in the presence of adjuvant as compared to mice immunized with non-adjuvanted Ssol. At the 2 μg and 0.2 μg antigen dose, the response was significantly (0.8-0.9 log 10) higher for mice immunized with the oil-in-water emulsion adjuvant (GSK2 adjuvant)-adjuvanted Ssol protein (p<0.005) as compared to mice immunized with the Alum-adjuvanted Ssol protein. A trend for higher antibody response was observed after immunization of mice with 0.02 μg Ssol adjuvanted with the oil-in-water emulsion adjuvant (GSK2 adjuvant) compared to mice immunized with alum-adjuvanted (p=0.09, and difference=0.2 log 10) or plain (p=0.02 Ssol, and difference=0.3 log 10) Ssol protein.

### Isotype Analysis of Anti-SARS-CoV Antibodies

In mice immunized either with the non-adjuvanted Ssol protein or with the Ssol protein adjuvanted with alum, the response was found to be strongly biased towards the IgG1 isotype whereas no or very low levels of IgG2b antibodies were detected (FIG. 16). In mice immunized with the oil-in-water emulsion adjuvant (GSK2 adjuvant)—adjuvanted Ssol protein, high titers of IgG1 (5.0±0.2 log 10 titers) antibodies were reached. Remarkably, titers of IgG2b (3.7±0.5 log 10 titers) antibodies albeit heterogeneous were significantly increased as compared to those observed in mice immunized with the Ssol protein adjuvanted with alum (1.7±0.04 log 10 titers , p<10⁻⁶).

### Neutralizing Antibodies

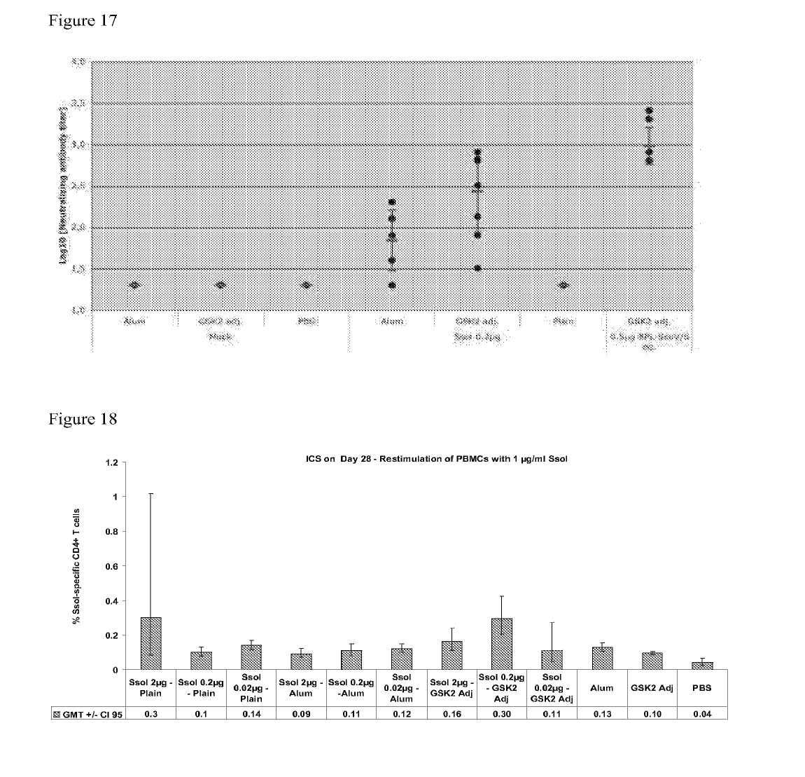

In mice immunized with the oil-in-water emulsion adjuvant (GSK2 adjuvant)-adjuvanted Ssol protein, neutralizing antibody titers (2.4±0.5 log 10 titers) were significantly higher than in mice immunized with the alum-adjuvanted Ssol protein (1.8±0.4 log 10 titers, p=0.02) whereas in mice immunized with the non-adjuvanted Ssol protein neutralizing titers fell below the detection limit (FIG. 17).

### B) Cellular Immune Response to Adjuvanted Ssol Protein in C57B1/6 Mice

The cell-mediated immune responses of the “Plain”, Alum-adjuvanted and oil-in-water emulsion (GSK2 adj)-adjuvanted groups of C57B1/6 mice were investigated as described in Example 3 for the BALB/c mice. However, due to a technical issue with spleen collection, no cellular responses in spleen were available for mice immunized with plain formulations (non-adjuvanted Ssol protein) or mice immunized with Alum-adjuvanted Ssol protein.

### CD4+ T Cell Responses in PBMC

Weak frequencies of CD4+ T cells were obtained in mice immunized with Ssol protein adjuvanted with GSK2 adjuvant or with Alum-adjuvanted Ssol, regardless of the dose (FIG. 18). Higher CD4+Tcell response was induced by 0.2 μg Ssol protein adjuvanted with GSK2 adjuvant compared to mice immunized with Alum-adjuvanted Ssol protein (with all three doses) or the non-adjuvanted Ssol protein (0.2 μg and 0.02 μg). Similar but weak frequencies were observed between the non-adjuvanted Ssol at 2 μg and the Ssol protein adjuvanted with GSK2 adjuvant at 0.2 μg (FIG. 18)

### CD4+ T Cell Responses in Spleen

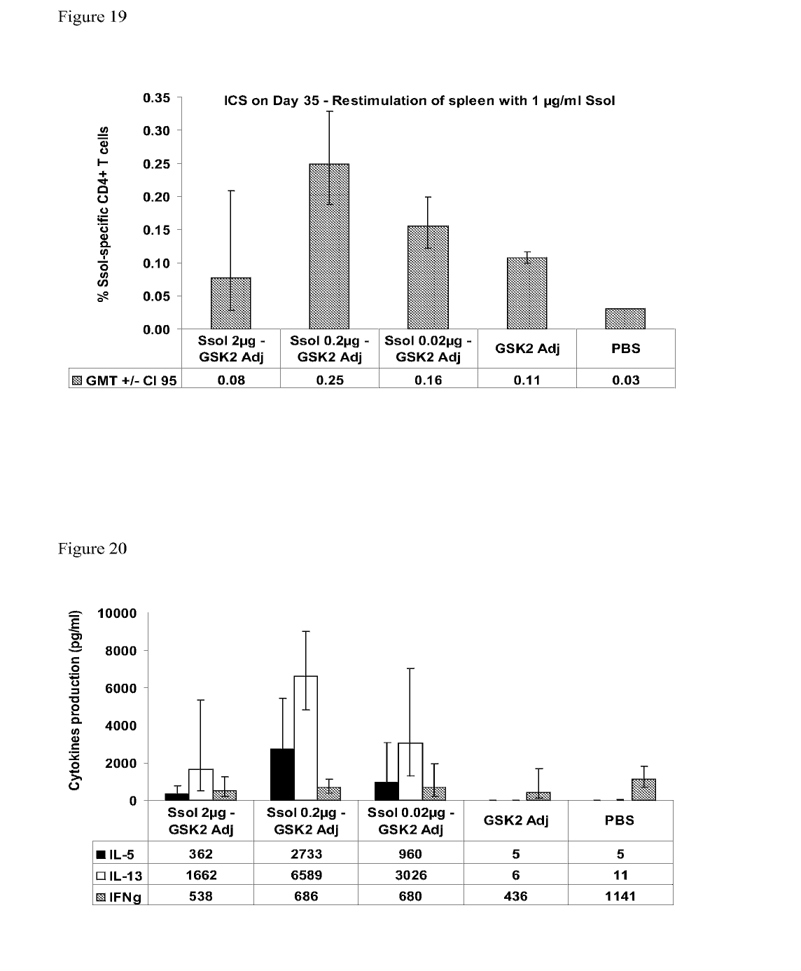

A trend for higher CD4+ T cell responses was observed after immunization of mice with 0.2 μg of Ssol protein adjuvanted with GSK2 adjuvant compared to mice immunized with 2 or 0.02 μg of Ssol protein adjuvanted with the same adjuvant (FIG. 19). Significantly higher (p<0.05) CD4+ T cell responses were observed after immunization of mice with 0.2 μg Ssol protein adjuvanted with GSK2 adjuvant compared to mice immunized with GSK2 adjuvant alone. The doses of 0.2 μg or 0.02 μg Ssol adjuvanted with GSK2 adjuvant induced similar level of CD4+ T cell responses (FIG. 19).

Cytokine Secretion from Spleen Cells

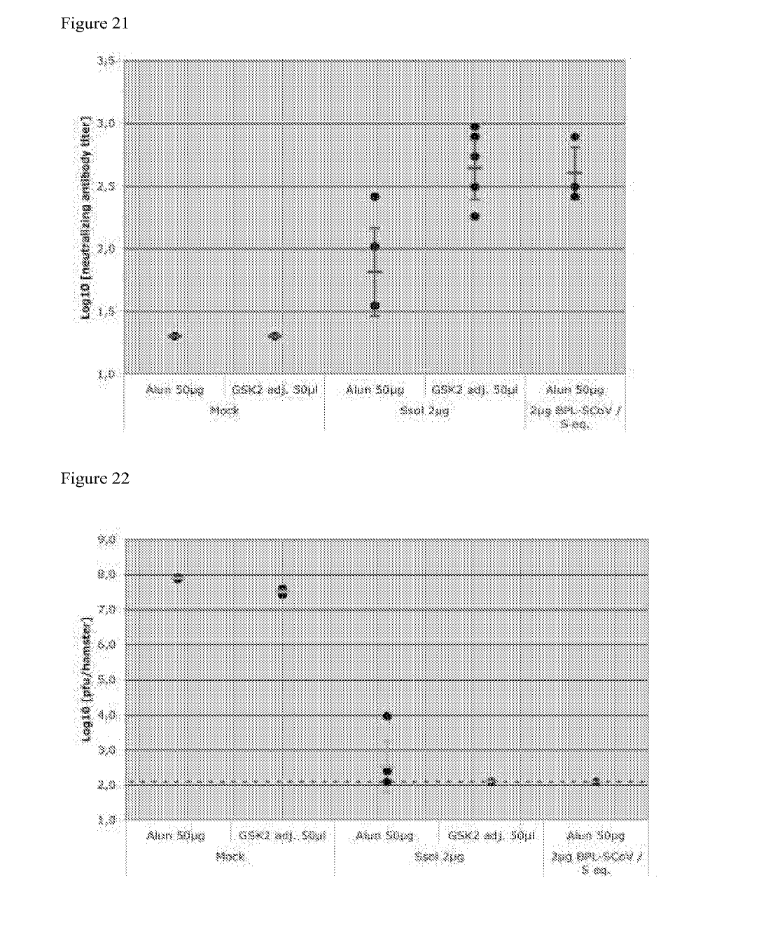

A trend for higher production of IL-5 and IL-13 cytokines was observed in mice immunized with 0.2 μg Ssol protein adjuvanted with GSK2 adjuvant compared to mice immunized with 2 or 0.02 μg Ssol protein adjuvanted with GSK2 adjuvant (FIG. 20). Whatever the dose of Ssol protein adjuvanted with GSK2 adjuvant, similar levels of IFN-γ cytokines were observed (FIG. 20).

### Summary of Results and Conclusions for Examples 3 and 4

These data demonstrated that in general the adjuvantation of Ssol protein with the oil-in-water emulsion adjuvant (GSK2 adjuvant) induced higher levels of anti-SARS-CoV ELISA antibody responses and neutralizing antibody responses in both BALB/c and C57BL/6 mice as compared to immunization with the Ssol protein either in the absence of adjuvant or in the presence of alum. Furthermore, the adjuvantation of the Ssol protein with GSK2 adjuvant induced higher CD4+ T cell responses compared to immunization with Alum-adjuvanted Ssol or the non-adjuvanted Ssol protein. The Ssol protein adjuvanted with GSK2 adjuvant provided a mixed Th1/Th2-like profile of the response as indicated by higher production of Th1 and Th2-type cytokines and an increased production of IgG2a or IgG2b in BALB/c and C57BL/6 mice, respectively.

```
Amino acid sequence of SARS-CoV #031589 strain S protein
SEQ ID NO: 1
MFIFLLFLTL TSGSDLDRCT TFDDVQAPNY TQHTSSMRGV YYPDEIFRSD TLYLTQDLFL PFYSNVTGFH
TINHTFGNPV IPFKDGIYFA ATEKSNVVRG WVFGSTMNNK SQSVIIINNS TNVVIRACNF ELCDNPFFAV
SKPMGTQTHT MIFDNAFNCT FEYISDAFSL DVSEKSGNFK HLREFVFKNK DGFLYVYKGY QPIDVVRDLP
SGFNTLKPIF KLPLGINITN FRAILTAFSP AQDIWGTSAA AYFVGYLKPT TFMLKYDENG TITDAVDCSQ
NPLAELKCSV KSFEIDKGIY QTSNFRVVPS GDVVRFPNIT NLCPFGEVFN ATKFPSVYAW ERKKISNCVA
DYSVLYNSTF FSTFKCYGVS ATKLNDLCFS NVYADSFVVK GDDVRQIAPG QTGVIADYNY KLPDDFMGCV
LAWNTRNIDA TSTGNYNYKY RYLRHGKLRP FERDISNVPF SPDGKPCTPP ALNCYWPLND YGFYTTTGIG
YQPYRVVVLS FELLNAPATV CGPKLSTDLI KNQCVNFNFN GLTGTGVLTP SSKRFQPFQQ FGRDVSDFTD
SVRDPKTSEI LDISPCSFGG VSVITPGTNA SSEVAVLYQD VNCTDVSTAI HADQLTPAWR IYSTGNNVFQ
TQAGCLIGAE HVDTSYECDI PIGAGICASY HTVSLLRSTS QKSIVAYTMS LGADSSIAYS NNTIAIPTNF
SISITTEVMP VSMAKTSVDC NMYICGDSTE CANLLLQYGS FCTQLNRALS GIAAEQDRNT REVFAQVKQM
YKTPTLKYFG GFNFSQILPD PLKPTKRSFI EDLLFNKVTL ADAGFMKQYG ECLGDINARD LICAQKFNGL
TVLPPLLTDD MIAAYTAALV SGTATAGWTF GAGAALQIPF AMQMAYRFNG IGVTQNVLYE NQKQIANQFN
KAISQIQESL TTTSTALGKL QDVVNQNAQA LNTLVKQLSS NFGAISSVLN DILSRLDKVE AEVQIDRLIT
GRLQSLQTYV TQQLIRAAEI RASANLAATK MSECVLGQSK RVDFCGKGYH LMSFPQAAPH GVVFLHVTYV
PSQERNFTTA PAICHEGKAY FPREGVFVFN GTSWFITQRN FFSPQIITTD NTFVSGNCDV VIGIINNTVY
DPLQPELDSF KEELDKYFKN HTSPDVDLGD ISGINASVVN IQKEIDRLNE VAKNLNESLI DLQELGKYEQ
YIKWPWYVWL GFIAGLIAIV MVTILLCCMT SCCSCLKGAC SCGSCCKFDE DDSEPVLKGV KLHYT
Ssol amino acid sequence
Amino acids 1-13 correspond to the signal peptide and are cleaved from the mature
protein (underlined). Ser-Gly linker and FLAG peptide sequences are in bold.
SEQ ID NO: 2
MFIFLLFLTL TSGSDLDRCT TFDDVQAPNY TQHTSSMRGV YYPDEIFRSD TLYLTQDLFL PFYSNVTGFH
TINHTFGNPV IPFKDGIYFA ATEKSNVVRG WVFGSTMNNK SQSVIIINNS TNVVIRACNF ELCDNPFFAV
SKPMGTQTHT MIFDNAFNCT FEYISDAFSL DVSEKSGNFK HLREFVFKNK DGFLYVYKGY QPIDVVRDLP
SGFNTLKPIF KLPLGINITN FRAILTAFSP AQDIWGTSAA AYFVGYLKPT TFMLKYDENG TITDAVDCSQ
NPLAELKCSV KSFEIDKGIY QTSNFRVVPS GDVVRFPNIT NLCPFGEVFN ATKFPSVYAW ERKKISNCVA
DYSVLYNSTF FSTFKCYGVS ATKLNDLCFS NVYADSFVVK GDDVRQIAPG QTGVIADYNY KLPDDFMGCV
LAWNTRNIDA TSTGNYNYKY RYLRHGKLRP FERDISNVPF SPDGKPCTPP ALNCYWPLND YGFYTTTGIG
YQPYRVVVLS FELLNAPATV CGPKLSTDLI KNQCVNFNFN GLTGTGVLTP SSKRFQPFQQ FGRDVSDFTD
SVRDPKTSEI LDISPCSFGG VSVITPGTNA SSEVAVLYQD VNCTDVSTAI HADQLTPAWR IYSTGNNVFQ
TQAGCLIGAE HVDTSYECDI PIGAGICASY HTVSLLRSTS QKSIVAYTMS LGADSSIAYS NNTIAIPTNF
SISITTEVMP VSMAKTSVDC NMYICGDSTE CANLLLQYGS FCTQLNRALS GIAAEQDRNT REVFAQVKQM
YKTPTLKYFG GFNFSQILPD PLKPTKRSFI EDLLFNKVTL ADAGFMKQYG ECLGDINARD LICAQKFNGL
TVLPPLLTDD MIAAYTAALV SGTATAGWTF GAGAALQIPF AMQMAYRFNG IGVTQNVLYE NQKQIANQFN
KAISQIQESL TTTSTALGKL QDVVNQNAQA LNTLVKQLSS NFGAISSVLN DILSRLDKVE AEVQIDRLIT
GRLQSLQTYV TQQLIRAAEI RASANLAATK MSECVLGQSK RVDFCGKGYH LMSFPQAAPH GVVFLHVTYV
PSQERNFTTA PAICHEGKAY FPREGVFVFN GTSWFITQRN FFSPQIITTD NTFVSGNCDV VIGIINNTVY
DPLQPELDSF KEELDKYFKN HTSPDVDLGD ISGINASVVN IQKEIDRLNE VAKNLNESLI DLQELGKYEQ
YIKSGDYKDD DDK
DNA sequence encoding S protein, inserted within a BamH1-Xho1 cassette, as in
pCI-S-WPRE). ATG and TAA codons are underlined, extra-sequences (BamH1,
Xho1, Kozak sequences are in bold).
SEQ ID NO: 3
      GGATCCA CCATGTTTAT TTTCTTATTA TTTCTTACTC TCACTAGTGG TAGTGACCTT GACCGGTGCA
CCACTTTTGA TGATGTTCAA GCTCCTAATT ACACTCAACA TACTTCATCT ATGAGGGGGG TTTACTATCC
TGATGAAATT TTTAGATCAG ACACTCTTTA TTTAACTCAG GATTTATTTC TTCCATTTTA TTCTAATGTT
ACAGGGTTTC ATACTATTAA TCATACGTTT GGCAACCCTG TCATACCTTT TAAGGATGGT ATTTATTTTG
CTGCCACAGA GAAATCAAAT GTTGTCCGTG GTTGGGTTTT TGGTTCTACC ATGAACAACA AGTCACAGTC
GGTGATTATT ATTAACAATT CTACTAATGT TGTTATACGA GCATGTAACT TTGAATTGTG TGACAACCCT
TTCTTTGCTG TTTCTAAACC CATGGGTACA CAGACACATA CTATGATATT CGATAATGCA TTTAATTGCA
CTTTCGAGTA CATATCTGAT GCCTTTTCGC TTGATGTTTC AGAAAAGTCA GGTAATTTTA AACACTTACG
AGAGTTTGTG TTTAAAAATA AAGATGGGTT TCTCTATGTT TATAAGGGCT ATCAACCTAT AGATGTAGTT
CGTGATCTAC CTTCTGGTTT TAACACTTTG AAACCTATTT TTAAGTTGCC TCTTGGTATT AACATTACAA
ATTTTAGAGC CATTCTTACA GCCTTTTCAC CTGCTCAAGA CATTTGGGGC ACGTCAGCTG CAGCCTATTT
TGTTGGCTAT TTAAAGCCAA CTACATTTAT GCTCAAGTAT GATGAAAATG GTACAATCAC AGATGCTGTT
GATTGTTCTC AAAATCCACT TGCTGAACTC AAATGCTCTG TTAAGAGCTT TGAGATTGAC AAAGGAATTT
ACCAGACCTC TAATTTCAGG GTTGTTCCCT CAGGAGATGT TGTGAGATTC CCTAATATTA CAAACTTGTG
TCCTTTTGGA GAGGTTTTTA ATGCTACTAA ATTCCCTTCT GTCTATGCAT GGGAGAGAAA AAAAATTTCT
AATTGTGTTG CTGATTACTC TGTGCTCTAC AACTCAACAT TTTTTTCAAC CTTTAAGTGC TATGGCGTTT
CTGCCACTAA GTTGAATGAT CTTTGCTTCT CCAATGTCTA TGCAGATTCT TTTGTAGTCA AGGGAGATGA
TGTAAGACAA ATAGCGCCAG GACAAACTGG TGTTATTGCT GATTATAATT ATAAATTGCC AGATGATTTC
ATGGGTTGTG TCCTTGCTTG GAATACTAGG AACATTGATG CTACTTCAAC TGGTAATTAT AATTATAAAT
ATAGGTATCT TAGACATGGC AAGCTTAGGC CCTTTGAGAG AGACATATCT AATGTGCCTT TCTCCCCTGA
TGGCAAACCT TGCACCCCAC CTGCTCTTAA TTGTTATTGG CCATTAAATG ATTATGGTTT TTACACCACT
ACTGGCATTG GCTACCAACC TTACAGAGTT GTAGTACTTT CTTTTGAACT TTTAAATGCA CCGGCCACGG
TTTGTGGACC AAAATTATCC ACTGACCTTA TTAAGAACCA GTGTGTCAAT TTTAATTTTA ATGGACTCAC
TGGTACTGGT GTGTTAACTC CTTCTTCAAA GAGATTTCAA CCATTTCAAC AATTTGGCCG TGATGTCTCT
GATTTCACTG ATTCCGTTCG AGATCCTAAA ACATCTGAAA TATTAGACAT TTCACCTTGC TCTTTTGGGG
GTGTAAGTGT AATTACACCT GGAACAAATG CTTCATCTGA AGTTGCTGTT CTATATCAAG ATGTTAACTG
CACTGATGTT TCTACAGCAA TCCATGCAGA TCAACTCACA CCAGCTTGGC GCATATATTC TACTGGAAAC
AATGTATTCC AGACTCAAGC AGGCTGTCTT ATAGGAGCTG AGCATGTCGA CACTTCTTAT GAGTGCGACA
TTCCTATTGG AGCTGGCATT TGTGCTAGTT ACCATACAGT TTCTTTATTA CGTAGTACTA GCCAAAAATC
TATTGTGGCT TATACTATGT CTTTAGGTGC TGATAGTTCA ATTGCTTACT CTAATAACAC CATTGCTATA
CCTACTAACT TTTCAATTAG CATTACTACA GAAGTAATGC CTGTTTCTAT GGCTAAAACC TCCGTAGATT
GTAATATGTA CATCTGCGGA GATTCTACTG AATGTGCTAA TTTGCTTCTC CAATATGGTA GCTTTTGCAC
ACAACTAAAT CGTGCACTCT CAGGTATTGC TGCTGAACAG GATCGCAACA CACGTGAAGT GTTCGCTCAA
GTCAAACAAA TGTACAAAAC CCCAACTTTG AAATATTTTG GTGGTTTTAA TTTTTCACAA ATATTACCTG
ACCCTCTAAA GCCAACTAAG AGGTCTTTTA TTGAGGACTT GCTCTTTAAT AAGGTGACAC TCGCTGATGC
TGGCTTCATG AAGCAATATG GCGAATGCCT AGGTGATATT AATGCTAGAG ATCTCATTTG TGCGCAGAAG
TTCAATGGGC TTACAGTGTT GCCACCTCTG CTCACTGATG ATATGATTGC TGCCTACACT GCTGCTCTAG
TTAGTGGTAC TGCCACTGCT GGATGGACAT TTGGTGCTGG CGCTGCTCTT CAAATACCTT TTGCTATGCA
AATGGCATAT AGGTTCAATG GCATTGGAGT TACCCAAAAT GTTCTCTATG AGAACCAAAA ACAAATCGCC
AACCAATTTA ACAAGGCGAT TAGTCAAATT CAAGAATCAC TTACAACAAC ATCAACTGCA TTGGGCAAGC
TGCAAGACGT TGTTAACCAG AATGCTCAAG CATTAAACAC ACTTGTTAAA CAACTTAGCT CTAATTTTGG
TGCAATTTCA AGTGTGCTAA ATGATATCCT TTCGCGACTT GATAAAGTCG AGGCGGAGGT ACAAATTGAC
AGGCTAATTA CAGGCAGACT TCAAAGCCTT CAAACCTATG TAACACAACA ACTAATCAGG GCTGCTGAAA
TCAGGGCTTC TGCTAATCTT GCTGCTACTA AAATGTCTGA GTGTGTTCTT GGACAATCAA AAAGAGTTGA
CTTTTGTGGA AAGGGCTACC ACCTTATGTC CTTCCCACAA GCAGCCCCGC ATGGTGTTGT CTTCCTACAT
GTCACGTATG TGCCATCCCA GGAGAGGAAC TTCACCACAG CGCCAGCAAT TTGTCATGAA GGCAAAGCAT
ACTTCCCTCG TGAAGGTGTT TTTGTGTTTA ATGGCACTTC TTGGTTTATT ACACAGAGGA ACTTCTTTTC
TCCACAAATA ATTACTACAG ACAATACATT TGTCTCAGGA AATTGTGATG TCGTTATTGG CATCATTAAC
AACACAGTTT ATGATCCTCT GCAACCTGAG CTTGACTCAT TCAAAGAAGA GCTGGACAAG TACTTCAAAA
ATCATACATC ACCAGATGTT GATCTTGGCG ACATTTCAGG CATTAACGCT TCTGTCGTCA ACATTCAAAA
AGAAATTGAC CGCCTCAATG AGGTCGCTAA AAATTTAAAT GAATCACTCA TTGACCTTCA AGAATTGGGA
AAATATGAGC AATATATTAA ATGGCCTTGG TATGTTTGGC TCGGCTTCAT TGCTGGACTA ATTGCCATCG
TCATGGTTAC AATCTTGCTT TGTTGCATGA CTAGTTGTTG CAGTTGCCTC AAGGGTGCAT GCTCTTGTGG
TTCTTGCTGC AAGTTTGATG AGGATGACTC TGAGCCAGTT CTCAAGGGTG TCAAATTACA TTACACATAA
CTCGAG

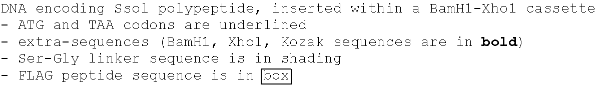

SEQ ID NO: 4
      GGATCCA CCATGTTTAT TTTCTTATTA TTTCTTACTC TCACTAGTGG TAGTGACCTT GACCGGTGCA
CCACTTTTGA TGATGTTCAA GCTCCTAATT ACACTCAACA TACTTCATCT ATGAGGGGGG TTTACTATCC
TGATGAAATT TTTAGATCAG ACACTCTTTA TTTAACTCAG GATTTATTTC TTCCATTTTA TTCTAATGTT
ACAGGGTTTC ATACTATTAA TCATACGTTT GGCAACCCTG TCATACCTTT TAAGGATGGT ATTTATTTTG
CTGCCACAGA GAAATCAAAT GTTGTCCGTG GTTGGGTTTT TGGTTCTACC ATGAACAACA AGTCACAGTC
GGTGATTATT ATTAACAATT CTACTAATGT TGTTATACGA GCATGTAACT TTGAATTGTG TGACAACCCT
TTCTTTGCTG TTTCTAAACC CATGGGTACA CAGACACATA CTATGATATT CGATAATGCA TTTAATTGCA
CTTTCGAGTA CATATCTGAT GCCTTTTCGC TTGATGTTTC AGAAAAGTCA GGTAATTTTA AACACTTACG
AGAGTTTGTG TTTAAAAATA AAGATGGGTT TCTCTATGTT TATAAGGGCT ATCAACCTAT AGATGTAGTT
CGTGATCTAC CTTCTGGTTT TAACACTTTG AAACCTATTT TTAAGTTGCC TCTTGGTATT AACATTACAA
ATTTTAGAGC CATTCTTACA GCCTTTTCAC CTGCTCAAGA CATTTGGGGC ACGTCAGCTG CAGCCTATTT
TGTTGGCTAT TTAAAGCCAA CTACATTTAT GCTCAAGTAT GATGAAAATG GTACAATCAC AGATGCTGTT
GATTGTTCTC AAAATCCACT TGCTGAACTC AAATGCTCTG TTAAGAGCTT TGAGATTGAC AAAGGAATTT
ACCAGACCTC TAATTTCAGG GTTGTTCCCT CAGGAGATGT TGTGAGATTC CCTAATATTA CAAACTTGTG
TCCTTTTGGA GAGGTTTTTA ATGCTACTAA ATTCCCTTCT GTCTATGCAT GGGAGAGAAA AAAAATTTCT
AATTGTGTTG CTGATTACTC TGTGCTCTAC AACTCAACAT TTTTTTCAAC CTTTAAGTGC TATGGCGTTT
CTGCCACTAA GTTGAATGAT CTTTGCTTCT CCAATGTCTA TGCAGATTCT TTTGTAGTCA AGGGAGATGA
TGTAAGACAA ATAGCGCCAG GACAAACTGG TGTTATTGCT GATTATAATT ATAAATTGCC AGATGATTTC
ATGGGTTGTG TCCTTGCTTG GAATACTAGG AACATTGATG CTACTTCAAC TGGTAATTAT AATTATAAAT
ATAGGTATCT TAGACATGGC AAGCTTAGGC CCTTTGAGAG AGACATATCT AATGTGCCTT TCTCCCCTGA
TGGCAAACCT TGCACCCCAC CTGCTCTTAA TTGTTATTGG CCATTAAATG ATTATGGTTT TTACACCACT
ACTGGCATTG GCTACCAACC TTACAGAGTT GTAGTACTTT CTTTTGAACT TTTAAATGCA CCGGCCACGG
TTTGTGGACC AAAATTATCC ACTGACCTTA TTAAGAACCA GTGTGTCAAT TTTAATTTTA ATGGACTCAC
TGGTACTGGT GTGTTAACTC CTTCTTCAAA GAGATTTCAA CCATTTCAAC AATTTGGCCG TGATGTCTCT
GATTTCACTG ATTCCGTTCG AGATCCTAAA ACATCTGAAA TATTAGACAT TTCACCTTGC TCTTTTGGGG
GTGTAAGTGT AATTACACCT GGAACAAATG CTTCATCTGA AGTTGCTGTT CTATATCAAG ATGTTAACTG
CACTGATGTT TCTACAGCAA TCCATGCAGA TCAACTCACA CCAGCTTGGC GCATATATTC TACTGGAAAC
AATGTATTCC AGACTCAAGC AGGCTGTCTT ATAGGAGCTG AGCATGTCGA CACTTCTTAT GAGTGCGACA
TTCCTATTGG AGCTGGCATT TGTGCTAGTT ACCATACAGT TTCTTTATTA CGTAGTACTA GCCAAAAATC
TATTGTGGCT TATACTATGT CTTTAGGTGC TGATAGTTCA ATTGCTTACT CTAATAACAC CATTGCTATA
CCTACTAACT TTTCAATTAG CATTACTACA GAAGTAATGC CTGTTTCTAT GGCTAAAACC TCCGTAGATT
GTAATATGTA CATCTGCGGA GATTCTACTG AATGTGCTAA TTTGCTTCTC CAATATGGTA GCTTTTGCAC
ACAACTAAAT CGTGCACTCT CAGGTATTGC TGCTGAACAG GATCGCAACA CACGTGAAGT GTTCGCTCAA
GTCAAACAAA TGTACAAAAC CCCAACTTTG AAATATTTTG GTGGTTTTAA TTTTTCACAA ATATTACCTG
ACCCTCTAAA GCCAACTAAG AGGTCTTTTA TTGAGGACTT GCTCTTTAAT AAGGTGACAC TCGCTGATGC
TGGCTTCATG AAGCAATATG GCGAATGCCT AGGTGATATT AATGCTAGAG ATCTCATTTG TGCGCAGAAG
TTCAATGGGC TTACAGTGTT GCCACCTCTG CTCACTGATG ATATGATTGC TGCCTACACT GCTGCTCTAG
TTAGTGGTAC TGCCACTGCT GGATGGACAT TTGGTGCTGG CGCTGCTCTT CAAATACCTT TTGCTATGCA
AATGGCATAT AGGTTCAATG GCATTGGAGT TACCCAAAAT GTTCTCTATG AGAACCAAAA ACAAATCGCC
AACCAATTTA ACAAGGCGAT TAGTCAAATT CAAGAATCAC TTACAACAAC ATCAACTGCA TTGGGCAAGC
TGCAAGACGT TGTTAACCAG AATGCTCAAG CATTAAACAC ACTTGTTAAA CAACTTAGCT CTAATTTTGG
TGCAATTTCA AGTGTGCTAA ATGATATCCT TTCGCGACTT GATAAAGTCG AGGCGGAGGT ACAAATTGAC
AGGCTAATTA CAGGCAGACT TCAAAGCCTT CAAACCTATG TAACACAACA ACTAATCAGG GCTGCTGAAA
TCAGGGCTTC TGCTAATCTT GCTGCTACTA AAATGTCTGA GTGTGTTCTT GGACAATCAA AAAGAGTTGA
CTTTTGTGGA AAGGGCTACC ACCTTATGTC CTTCCCACAA GCAGCCCCGC ATGGTGTTGT CTTCCTACAT
GTCACGTATG TGCCATCCCA GGAGAGGAAC TTCACCACAG CGCCAGCAAT TTGTCATGAA GGCAAAGCAT
ACTTCCCTCG TGAAGGTGTT TTTGTGTTTA ATGGCACTTC TTGGTTTATT ACACAGAGGA ACTTCTTTTC
TCCACAAATA ATTACTACAG ACAATACATT TGTCTCAGGA AATTGTGATG TCGTTATTGG CATCATTAAC
AACACAGTTT ATGATCCTCT GCAACCTGAG CTTGACTCAT TCAAAGAAGA GCTGGACAAG TACTTCAAAA
ATCATACATC ACCAGATGTT GATCTTGGCG ACATTTCAGG CATTAACGCT TCTGTCGTCA ACATTCAAAA
AGAAATTGAC CGCCTCAATG AGGTCGCTAA AAATTTAAAT GAATCACTCA TTGACCTTCA AGAATTGGGA

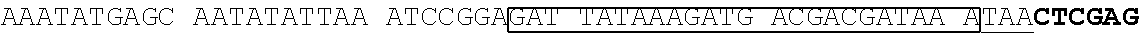

```

1. An immunogenic composition comprising:
(a) an immunogenic SARS coronavirus S (spike) polypeptide; and
(b) an oil-in-water emulsion adjuvant which comprises a metabolisable oil and an emulsifying agent.

2. The composition of claim 1, wherein the S polypeptide comprises the extracellular domain of the S protein.

3. The composition of claim 2, wherein the S polypeptide comprises amino acids 14 to 1193 of the SARS-CoV S protein fused at the C-terminus of the SARS-CoV S protein to the sequence SGDYKDDDDK (SEQ ID NO: 6).

4. The composition of claim 3, wherein the S polypeptide comprises the sequence of SEQ ID NO: 2.

5. The composition of claim 1, wherein the S polypeptide is present in an amount of from 1 to 5 μg per human dose.

6. The composition of claim 1, wherein the metabolisable oil is squalene.

7. The composition of claim 1, wherein the emulsifying agent is polyoxyethylene sorbitan monooleate (TWEEN™ 80).

8. The composition of claim 1, wherein the oil-in-water emulsion adjuvant further comprises a tocol.

9. The composition of claim 8, wherein the tocol is alpha-tocopherol.

10. The composition of claim 8, wherein the oil-in-water emulsion adjuvant comprises squalene, polyoxyethylene sorbitan monooleate (TWEEN™ 80) and alpha-tocopherol.

11. A method of producing the composition of claim 1, the method comprising combining an immunogenic SARS coronavirus S (spike) polypeptide with an oil-in-water emulsion adjuvant.

12-14. (canceled)

15. A method of preventing or treating severe acute respiratory syndrome or other SARS-CoV-related disease, which method comprises administering to 18 and an individual in need thereof an effective amount of an immunogenic composition comprising:
(a) an immunogenic SARS coronavirus S (spike) polypeptide; and
(b) an oil-in-water emulsion adjuvant which comprises a metabolisable oil and an emulsifying agent.

16. The method of claim 15, wherein the composition is administered in a single-dose vaccination schedule.

17. (canceled)

18. The immunogenic composition of claim 1, wherein the S polypeptide is an immunogenic fragment of a polypeptide having SEQ ID NO:1.

19. The immunogenic composition of claim 1, wherein the S polypeptide is a polypeptide having 95% sequence identity to SEQ ID NO:1.

20. The method of claim 15 wherein the S polypeptide is selected from the group consisting of SEQ ID NO: 1, an immunogenic fragment of a polypeptide having SEQ ID NO:1, and a polypeptide having 95% sequence identity to SEQ ID NO:1.
